(19) United States
(12) Patent Application Publication
(10) Pub. No.: US 2011/0229512 A1
(43) Pub. Date: Sep. 22, 2011

(54) COMPOSITIONS AND METHODS FOR DIAGNOSING AND TREATING URINARY TRACT INFECTIONS

(75) Inventor: Harry L.T. Mobley, Ann Arbor, MI (US)

(73) Assignee: THE REGENTS OF THE UNIVERSITY OF MICHIGAN, Ann Arbor, MI (US)

(21) Appl. No.: 13/051,134

(22) Filed: Mar. 18, 2011

Related U.S. Application Data
(60) Provisional application No. 61/315,456, filed on Mar. 19, 2010.

(51) Int. Cl. A61K 39/385; A61K 39/108; A61P 31/04; A61P 37/04

(52) U.S. Cl. 424/196.11; 424/257.1; 424/197.11

(57) ABSTRACT

The present invention relates to methods and compositions for treating urinary tract infections. In particular, the present invention relates to vaccines and immune modulators for treating urinary tract infections.

This application claims priority to provisional application Ser. No. 61/315,456, filed Mar. 19, 2010, which is herein incorporated by reference in its entirety.

This Application was supported by grant number AI043363 awarded by the National Institutes of Health. The government has certain rights in the invention.

### FIELD OF THE INVENTION

The present invention relates to methods and compositions for treating urinary tract infections. In particular, the present invention relates to vaccines and immune modulators for treating urinary tract infections.

### BACKGROUND OF THE INVENTION

A urinary tract infection (UTI) is an infection that begins in the urinary system. Serious consequences can occur if the infection spreads to the kidneys. Women are most at risk of developing a UTI. In fact, half of all women will develop a UTI during their lifetimes, and many will experience more than one. When treated promptly and properly, UTIs rarely lead to complications. But left untreated, a urinary tract infection can become something more serious than a set of uncomfortable symptoms. Untreated UTIs can lead to acute or chronic kidney infections (pyelonephritis), which could permanently damage kidneys. Young children and older adults are at the greatest risk of kidney damage due to UTIs because their symptoms are often overlooked or mistaken for other conditions. Women who have UTIs while pregnant may also have an increased risk of delivering low birth weight or premature infants. UTIs are generally treated with antibiotics as a first line of treatment. Drugs most commonly recommended for simple UTIs include amoxicillin (Amoxil, Trimox), nitrofurantoin (Furadantin, Macrodantin), trimethoprim (Proloprim) and the antibiotic combination of trimethoprim and sulfamethoxazole (Bactrim, Septra). For severe UTIs, hospitalization and treatment with intravenous antibiotics may be necessary. However, many antibiotic resistant bacteria are present in the environment, especially in hospital and other health care settings. Thus, additional treatments for UTIs are needed.

### SUMMARY OF THE INVENTION

The present invention relates to methods and compositions for treating urinary tract infections. In particular, the present invention relates to vaccines and immune modulators for treating urinary tract infections.

For example, in some embodiments, the present invention provides a vaccine composition comprising at least a portion of one or more immunogens (e.g., TosA and/or FyuA). In some embodiments, the vaccine composition further comprises one or more immunogens selected from, for example, Iha, IreA, IroN, lutA, TosA, FyuA or c2482 (e.g., extracellular loop 7 of IroN or loop 6 of IutA). In some embodiments, the immunogen is covalently bound to a carrier protein. The present invention is not limited to a particular carrier protein. Examples include, but are not limited to, exotoxin A, toxoids, virus like particles, tetanus toxin/toxoid, diphtheria toxin/toxoid or hepatitis B surface protein. In some embodiments, the carrier protein is a sterile pharmaceutically acceptable carrier protein. In some embodiments, the compositions are contained in a kit with additional components useful, necessary or sufficient for use of the vaccine composition (e.g., one or more of a device for administration of the vaccine, sanitation components, temperature control components, adjuvants, a physiologically tolerable buffer and instructions for using the vaccine composition).

In further embodiments, the present invention provides a method of inducing an immune response, comprising administering a composition comprising an effective amount of at least a portion of a TosA and/or FyuA immunogen to a subject under conditions such that the subject generates an immune response to a bacteria in the subjects urinary tract. In some embodiments, the immunogen is covalently bound to a carrier protein. In some embodiments, the bacteria are E. coli. In some embodiments, the immune response protects the subject from developing symptoms of a urinary tract infection. In some embodiments, the subject exhibits decreased levels of bacteria in the subject's bladder or kidney.

In yet other embodiments, the present invention provides a method of preventing urinary tract infections in a subject, comprising administering a composition comprising an effective amount of at least a portion of a Tos A and/or FyuA immunogen to a subject under conditions such that the subject does not develop symptoms of a urinary tract infection. In some embodiments, the subject exhibits decreased levels of bacteria in the subject's bladder or kidney.

Additional embodiments of the invention are described herein.

### DESCRIPTION OF THE FIGURES

FIG. 1 shows (A) Thirteen genomic islands of 30 kb in E. coli CFT073. (B) Confirmation of homologous recombination and subsequent replacement of each GI with a kanamycin resistance cassette.

FIG. 2 shows colonization levels in the bladder and kidneys of CBA/J mice at 48 hpi. Cochallenge of wild-type CFT073 with mutant strains or ΔPAI-aspV (n=20), ΔPAI-metV (n=14), and ΔPAI-asnT(n=10) in the bladder (A) and kidneys (B). Independent challenges of wild-type CFT073 (n=13) with mutant strains ΔPAI-aspV (n=11), ΔPAI-metV (n=10), and ΔPAI-asnT (n=10) in the bladder (C) and kidneys (D).

FIG. 3 shows colonization levels in the bladder (A) and kidneys (B) of CBA/J mice at 48 hpi during cochallenge of wild-type CFT073 with mutant strains ΔPAI-aspV1 (n=10), ΔPAI-aspV2 (n=10), ΔPAIaspV3 (n=9), and ΔPAI-aspV4 (n=10).

FIG. 4 shows colonization levels in the bladder (A) and kidneys (B) of CBA/J mice at 48 hpi during cochallenge of strains with mutations in individual genes of known function against the entire PAI mutant in which the gene is located.

FIG. 6 shows (A) Colonization levels in the bladder and kidneys of CBA/J mice at 48 hpi during cochallenge of wild-type CFT073 with the double iron system Δc0294-97 Δc2518-15 (Δfbp) mutant (n=16). The colonization levels in bladders (squares) and kidneys (circles) are indicated. (B) Growth of wild-type CFT073, Δc0294-97 Δc2518-15 (Δfbp) mutant, and an enterobactin/aerobactin-negative (entF::kan iucB::cam) strain of CFT073 on CAS siderophore agar. (C) Expression of the cloned fbp locus under the control of a constitutive em7 promoter (E. coli TOP10/pGENfbp) and the vector-only negative control (pGENMCS).

FIG. 7 shows colonization levels in the bladder (A) and kidneys (B) of CBA/J mice at 48 hpi during cochallenge of wild-type CFT073 with the ΔPAI-met V1 mutant (n=9) and ΔPAI-metV2 mutant (n=9).

FIG. 8 shows colonization levels in the bladder and kidneys of CBA/J mice at 48 hpi during cochallenge of wild-type CFT073 with the Δc3405-10 mutant (n=9). The colonization levels in bladders (squares) and kidneys (circles) are indicated.

FIG. 9 shows colonization levels in the bladder and kidneys of CBA/J mice 48 hr after cochallenge with wildtype CFT073 and Δc0363 (tosA/RTX) mutant (n=14).

FIG. 10 shows correlation of virulence gene expression by E. coli CFT073 in the murine model with the UPEC strains in the urine of women with clinical UTI.

FIG. 11 shows the polypeptide sequence of TosA (c0363) (SEQ ID NO:3).

FIG. 12 shows affinity purification of the FyuA antigen.

FIG. 13 shows FyuA immunization and Ec536 challenge.

### DEFINITIONS

To facilitate understanding of the invention, a number of terms are defined below.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “peptide” refers to a polymer of two or more amino acids joined via peptide bonds or modified peptide bonds. As used herein, the term “dipeptides” refers to a polymer of two amino acids joined via a peptide or modified peptide bond.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

As used herein, the term “purified” or “to purify” refers to the removal of contaminants from a sample. For example, antigens are purified by removal of contaminating proteins. The removal of contaminants results in an increase in the percent of antigen (e.g., antigen of the present invention) in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

As used herein, the term “microorganism” refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

As used herein the term “pathogen,” and grammatical equivalents, refers to an organism (e.g., biological agent), including microorganisms, that causes a disease state (e.g., infection, pathologic condition, disease, etc.) in another organism (e.g., animals and plants) by directly infecting the other organism, or by producing agents that causes disease in another organism (e.g., bacteria that produce pathogenic toxins and the like). “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a bacteria)), may be responsive to environmental factors (e.g., malnutrition, industrial hazards, and/or climate), may be responsive to an inherent defect of the organism (e.g., genetic anomalies) or to combinations of these and other factors.

The term “emulsion,” as used herein, includes classic oil-in-water or water in oil dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in preferred embodiments, the nanoemulsions comprise an oil phase having droplets with a mean particle size of approximately 0.1 to 5 microns (e.g., 150+/−25 nm in diameter), although smaller and larger particle sizes are contemplated. The terms “emulsion” and “nanoemulsion” are often used herein, interchangeably, to refer to the nanoemulsions of the present invention.

As used herein, the terms “contact,” “contacted,” “expose,” and “exposed,” when used in reference to a nanoemulsion and a live microorganism, refer to bringing one or more nanoemulsions into contact with a microorganism (e.g., a pathogen) such that the nanoemulsion inactivates the microorganism or pathogenic agent, if present. The present invention is not limited by the amount or type of nanoemulsion used for microorganism inactivation. A variety of nanoemulsion that find use in embodiments of the present invention are described herein and elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes). Ratios and amounts of nanoemulsion (e.g., sufficient for inactivating the microorganism (e.g., bacterial inactivation)) and microorganisms (e.g., sufficient to provide an antigenic composition (e.g., a composition capable of inducing an immune response)) are contemplated in the present invention including, but not limited to, those described herein.

As used herein, the term “adjuvant” refers to any substance that can stimulate an immune response (e.g., a mucosal immune response). Some adjuvants can cause activation of a cell of the immune system (e.g., an adjuvant can cause an immune cell to produce and secrete a cytokine). Examples of adjuvants that can cause activation of a cell of the immune system include, but are not limited to, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present invention (e.g., comprising nanoemulsion inactivated RSV) are administered with one or more adjuvants (e.g., to skew the immune response towards a Th1 or Th2 type response).

As used herein; the term “carrier protein” references to a molecule that interacts (e.g., via covalent attachment) to an antigen or immunogen. In some embodiments, carrier proteins enhance the presentation of antigens to the immune system. In some embodiments, the resulting complex engages the T cell arm of the immune response, resulting in higher levels of antibodies and cell response. In some embodiments, carrier proteins are sterile and pharmaceutically acceptable.

As used herein, the term “under conditions such that said subject generates an immune response” refers to any qualitative or quantitative induction, generation, and/or stimulation of an immune response (e.g., innate or acquired).

A used herein, the term “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired (e.g., immune responses that are mediated by B and T cells following a previous exposure to antigen (e.g., that exhibit increased specificity and reactivity to the antigen)).

As used herein, the term “immunogen” refers to an agent (e.g., a microorganism (e.g., bacterium, virus or fungus) and/or portion or component thereof (e.g., a protein antigen)) that is capable of eliciting an immune response in a subject. In some embodiments, immunogens elicit immunity against the immunogen (e.g., microorganism (e.g., pathogen or a pathogen product)) when administered in combination with a nanoemulsion.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. As used herein, the term “sample” is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

### DETAILED DESCRIPTION OF THE INVENTION

The present invention relates to methods and compositions for treating urinary tract infections. In particular, the present invention relates to vaccines and immune modulators for treating urinary tract infections.

Uropathogenic Escherichia coli (UPEC) are responsible for the majority of uncomplicated urinary tract infections, which can present clinically as cystitis or pyelonephritis. UPEC strain CFT073, isolated from the blood of a patient with acute pyelonephritis, is a highly cytotoxic and virulent strain.

Uropathogenic E. coli strains, as exemplified by strain CFT073, have acquired pathogenicity islands that contribute to fitness in the urinary tract. During experiments conducted during the course of development of the present invention, three of the nine genomic island deletion mutants tested were significantly outcompeted by wild-type CFT073 in the bladders or kidneys of CBA/J mice during experimental cochallenge. The PAI-aspV and PAI-metV mutants showed attenuation in the ability to colonize bladders and kidneys, while the PAI-asnT mutant showed attenuation in the kidneys only. In contrast, all mutants were able to colonize the urinary tracts of mice to levels similar to that of the wild-type strain during independent challenges of mice with the exception of the PAI-metV mutant, which was attenuated in the kidneys (and tended toward attenuation in the bladder [P=0.0668]). The ability of these mutants to colonize the murine urinary tract to a level comparable to that of the wild type when not in a competitive setting demonstrates that while the genes contained in PAIs contribute to the overall fitness of the strain, they are generally not essential for survival in the host.

The pangenome of E. coli was recently described by Rasko and colleagues (Rasko et al., 2008. J. Bacteriol. 190:6881-6893). Analysis of 17 sequenced E. coli genomes, including commensal and pathogenic strains, revealed the genome size of E. coli to be 5,020±446 genes (mean ±standard deviation) with a “conserved core” genome size (genes that are highly conserved in all 17 isolates) of 2,344±43 genes (Rasko et al., 2008. J. Bacteriol. 190:6881-6893).

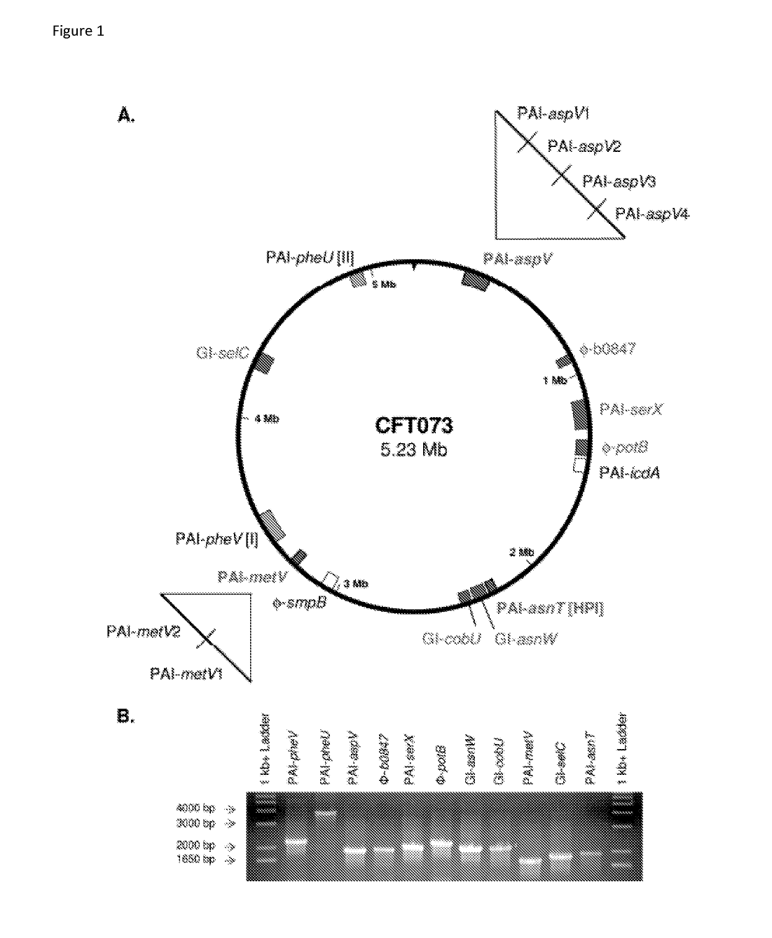

E. coli CFT073 has the largest genome (5.23 Mb) of the three sequenced and annotated UPEC strains and is 592 kb larger than E. coli K-12 (Welch et al., 2002. Proc. Natl. Acad. Sci. USA 99:17020-17024). It is estimated that 17 to 18% of all ORFs in E. coli strain K-12 MG1655 were horizontally acquired (Lawrence et al., 1998. Proc. Natl. Acad. Sci. USA 95:9413-9417; Nakamura et al., 2004. Nat. Genet. 36:760-766). Pathogenic strains of E. coli generally have larger genomes than commensal E. coli isolates, with the majority of this difference attributed to the insertion of relatively few large chromosomal regions (Rode et al., 1999. Infect. Immun. 67:230-236). These GIs are acquired by horizontal gene transfer (Ochman et al., 2000. Nature 405:299-304) and comprise 12.8% of the CFT073 genome (Lloyd et al., 2007. J. Bacteriol. 189:3532-3546). GIs contribute to bacterial fitness (Hacker and Carniel. 2001. EMBO Rep. 2:376-381) by conferring new properties that increase the adaptability of the organism and may also encode genes involved in pathogenicity. Strain CFT073 has 13 genomic or pathogenicity islands (FIG. 1).

Individual deletion of three PAIs attenuated the ability of wild-type strain CFT073 to colonize in cochallenge with the wild-type strain. PAI-aspV showed a median level of colonization that was about 0.5 log unit less than CFT073 in the bladder and 2 log units less in the kidneys. Construction of mutants in smaller regions of two PAIs allowed for the localization of the observed attenuation phenotype to specific genes or operons, often with organ-specific effects. For example, the aspV PAI was further characterized by construction of four mutants, each deleting smaller sections of the PAI (PAI-aspVI, PAI-aspV2, PAI-aspV3, and PAIaspV4) (FIG. 1). Testing the smaller mutants in cochallenge revealed that PAIaspV3, containing the contact-dependent inhibition gene cdiA and the autotransporter protease gene picU, was important for colonization of the bladder, whereas PAI-aspV4, containing the RTX family exoprotein A gene, c0363, previously described as tosA for type 1 secretion (Parham et al., 2005. J. Clin. Microbiol. 43:2425-2434), was significantly outcompeted in the kidneys of mice.

Testing mutants in three genes of known function located within PAI-aspV in cochallenge against the whole-island mutant demonstrated an organ-specific phenotype for two of the mutants, indicating that some genes play a more crucial role in either the bladder or the kidneys, but not necessarily in both organs. Mutants with deletions in picU and c0294-97 showed levels of colonization in the bladder similar to that of the PAI-aspV whole-island mutant, indicating that these two genes are at least partially responsible for the attenuation observed in the bladder with the ΔPAI-aspV mutant. However, neither mutant was attenuated in their ability to colonize the kidneys. Deletion of c0363, the putative RTX toxin (tosA) gene resulted in statistically less colonization than wild-type CFT073 in both the bladder and kidneys of mice at 48 hpi despite this trend not reaching significance in the bladder for the PAI-aspV4 mutant lacking the c0363 gene.

The present invention is not limited to a particular antigen. Exemplary antigens include, but are not limited to, TosA (c0363), Iha (iron-regulated gene homolog adhesin), IreA (iron-responsive element), IVIAT proteins, FyuA (putative pesticin receptor), Hma (c2482 (novel heme-binding protein)), and cytoplasmic proteins upregulated in urine. In other embodiments, the antigen is ChuA, IroN, or IutA. Exemplary antigens are described, for example, in U.S. Patent Publication 2009-0068220, herein incorporated by reference in its entirety. In some embodiments, the proteins identified in Example 1 are utilized. Additional antigens are known to those of skill in the art. In some embodiments, one or more antigens are used in combination. The present invention is not limited to a particular combination of antigens. In some embodiments, one or more, two or more, three or more, or a larger number of antigens are administered in combination.

In some embodiments, fragments of antigens are utilized for immunization. For example, in some embodiments, extracellular domain or loops (e.g., loop 7 of IroN or loop 6 of IutA) are utilized as antigens.

In some embodiments, immunocarriers (e.g., carrier proteins) are attached to immunogens (e.g., sterile pharmaceutically acceptable carrier proteins). In some embodiments, carrier proteins are covalently linked to an antigen or immunogen. Exemplary carrier proteins include, but are not limited to, cholera toxin, pseudomonas exotoxin A, toxoids, virus like particles, tetanus toxin/toxoid, diphtheria toxin/toxoid and hepatitis B surface protein. Additional carrier proteins are known to those of skill in the art.

An effective amount of the present vaccine is one in which a sufficient immunological response to the vaccine is raised to protect a subject exposed to bacteria in the urinary tract (e.g., E. coli) from contracting a UTI. Preferably, the subject is protected to an extent in which from one to all of the adverse physiological symptoms or effects (e.g., excess bacteria in the urinary tract, inflammation, and pain) of the disease to be prevented are found to be significantly reduced.

In some embodiments, the present invention provides compositions for inducing immune responses comprising a nanoemulsion (See e.g., WO 09/143524; 10/148111 and 10/057197; each of which is herein incorporated by reference in its entirety).The present invention is not limited to any particular nanoemulsion. Indeed, a variety of nanoemulsions find use in the invention including, but not limited to, those described herein and those described elsewhere (e.g., nanoemulsions described in U.S. Pat. Apps. 20020045667 and 20040043041, and U.S. Pat. Nos. 6,015,832, 6,506,803, 6,635,676, and 6,559,189, each of which is incorporated herein by reference in its entirety for all purposes).

Furthermore, in some embodiments, a composition of the present invention induces (e.g., when administered to a subject) both systemic and mucosal immunity. Thus, in some preferred embodiments, administration of a composition of the present invention to a subject results in protection against an exposure to pathogenic E. coli.

In some embodiments, the present invention provides a composition comprising a nanoemulsion and an E. coli immunogen (e.g., those described herein) to serve as a mucosal vaccine. In some embodiments, this material can easily be produced. The ability to produce this formulation rapidly and administer it via mucosal (e.g., nasal) instillation provides a vaccine that can be used in large-scale administrations (e.g., to a population of a town, village, city, state or country).

In some embodiments, the present invention provides a composition for generating an immune response comprising a nanoemulsion and an E. coli immunogen (e.g., those described herein). When administered to a subject, a composition of the present invention stimulates an immune response against the immunogen within the subject. Although an understanding of the mechanism is not necessary to practice the present invention and the present invention is not limited to any particular mechanism of action, in some embodiments, generation of an immune response (e.g., resulting from administration of a composition comprising a nanoemulsion and an immunogen) provides total or partial immunity to the subject (e.g., from signs, symptoms or conditions of a disease (e.g., UTI)). Without being bound to any specific theory, protection and/or immunity from disease (e.g., the ability of a subject's immune system to prevent or attenuate (e.g., suppress) a sign, symptom or condition of disease) after exposure to an immunogenic composition of the present invention is due to adaptive (e.g., acquired) immune responses (e.g., immune responses mediated by B and T cells following exposure to an immunogen (e.g., immune responses that exhibit increased specificity and reactivity towards the immunogens described herein). Thus, in some embodiments, the compositions and methods of the present invention are used prophylactically or therapeutically to prevent or attenuate a sign, symptom or condition associated with a UTI.

In some embodiments, a composition comprising a nanoemulsion and an immunogen is administered alone. In some embodiments, a composition comprising a nanoemulsion and a immunogen comprises one or more other agents (e.g., a pharmaceutically acceptable carrier, adjuvant, carrier protein, excipient, and the like). In some embodiments, a composition for stimulating an immune response of the present invention is administered in a manner to induce a humoral immune response. In some embodiments, a composition for stimulating an immune response of the present invention is administered in a manner to induce a cellular (e.g., cytotoxic T lymphocyte) immune response, rather than a humoral response. In some embodiments, a composition comprising a nanoemulsion and an immunogen induces both a cellular and humoral immune response.

The present invention is not limited by the particular formulation of a composition comprising a nanoemulsion and a immunogen. Indeed, a composition comprising a nanoemulsion and an immunogen of the present invention may comprise one or more different agents in addition to the nanoemulsion and immunogen. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion and an immunogen of the present invention comprises an agent and/or co-factor that enhance the ability of the immunogen to induce an immune response (e.g., an adjuvant). In some preferred embodiments, the presence of one or more co-factors or agents reduces the amount of immunogen required for induction of an immune response (e.g., a protective immune respone (e.g., protective immunization)). In some embodiments, the presence of one or more co-factors or agents can be used to skew the immune response towards a cellular (e.g., T cell mediated) or humoral (e.g., antibody mediated) immune response. The present invention is not limited by the type of co-factor or agent used in a therapeutic agent of the present invention.

Adjuvants are described in general in Vaccine Design—the Subunit and Adjuvant Approach, edited by Powell and Newman, Plenum Press, New York, 1995. The present invention is not limited by the type of adjuvant utilized (e.g., for use in a composition (e.g., pharmaceutical composition). For example, in some embodiments, suitable adjuvants include an aluminium salt such as aluminium hydroxide gel (alum) or aluminium phosphate. In some embodiments, an adjuvant may be a salt of calcium, iron or zinc, or may be an insoluble suspension of acylated tyrosine, or acylated sugars, cationically or anionically derivatised polysaccharides, or polyphosphazenes.

In general, an immune response is generated to an antigen through the interaction of the antigen with the cells of the immune system. Immune responses may be broadly categorized into two categories: humoral and cell mediated immune responses (e.g., traditionally characterized by antibody and cellular effector mechanisms of protection, respectively). These categories of response have been termed Thl-type responses (cell-mediated response), and Th2-type immune responses (humoral response).

Stimulation of an immune response can result from a direct or indirect response of a cell or component of the immune system to an intervention (e.g., exposure to an immunogen). Immune responses can be measured in many ways including activation, proliferation or differentiation of cells of the immune system (e.g., B cells, T cells, dendritic cells, APCs, macrophages, NK cells, NKT cells etc.); up-regulated or down-regulated expression of markers and cytokines; stimulation of IgA, IgM, or IgG titer; splenomegaly (including increased spleen cellularity); hyperplasia and mixed cellular infiltrates in various organs. Other responses, cells, and components of the immune system that can be assessed with respect to immune stimulation are known in the art.

Although an understanding of the mechanism is not necessary to practice the present invention and the present invention is not limited to any particular mechanism of action, in some embodiments, compositions and methods of the present invention induce expression and secretion of cytokines (e.g., by macrophages, dendritic cells and CD4+ T cells). Modulation of expression of a particular cytokine can occur locally or systemically. It is known that cytokine profiles can determine T cell regulatory and effector functions in immune responses. In some embodiments, Th1-type cytokines can be induced, and thus, the immunostimulatory compositions of the present invention can promote a Th1 type antigen-specific immune response including cytotoxic T-cells (e.g., thereby avoiding unwanted Th2 type immune responses (e.g., generation of Th2 type cytokines (e.g., IL-13) involved in enhancing the severity of disease (e.g., IL-13 induction of mucus formation))).

Cytokines play a role in directing the T cell response. Helper (CD4+) T cells orchestrate the immune response of mammals through production of soluble factors that act on other immune system cells, including B and other T cells. Most mature CD4+T helper cells express one of two cytokine profiles: Th1 or Th2. Th1-type CD4+ T cells secrete IL-2, IL-3, IFN-γ, GM-CSF and high levels of TNF-α Th2 cells express IL-3, IL-4, IL-5, IL-6, IL-9, IL-10, IL-13, GM-CSF and low levels of TNF-α. Th1 type cytokines promote both cell-mediated immunity, and humoral immunity that is characterized by immunoglobulin class switching to IgG2a in mice and IgG1 in humans. Th1 responses may also be associated with delayed-type hypersensitivity and autoimmune disease. Th2 type cytokines induce primarily humoral immunity and induce class switching to IgG1 and IgE. The antibody isotypes associated with Th1 responses generally have neutralizing and opsonizing capabilities whereas those associated with Th2 responses are associated more with allergic responses.

Several factors have been shown to influence skewing of an immune response towards either a Th1 or Th2 type response. The best characterized regulators are cytokines. IL-12 and IFN-γ are positive Th1 and negative Th2 regulators. IL-12 promotes IFN-γ production, and IFN-γ provides positive feedback for IL-12. IL-4 and IL-10 appear important for the establishment of the Th2 cytokine profile and to down-regulate Th1 cytokine production. Thus, in preferred embodiments, the present invention provides a method of stimulating a Th1-type immune response in a subject comprising administering to a subject a composition comprising an immunogen. However, in other embodiments, the present invention provides a method of stimulating a Th2-type immune response in a subject (e.g., if balancing of a T cell mediated response is desired) comprising administering to a subject a composition comprising an immunogen. In further preferred embodiments, adjuvants can be used (e.g., can be co-administered with a composition of the present invention) to skew an immune response toward either a Th1 or Th2 type immune response. For example, adjuvants that induce Th2 or weak Th1 responses include, but are not limited to, alum, saponins, and SB-As4. Adjuvants that induce Th1 responses include but are not limited to MPL, MDP, 1SCOMS, IL-12, IFN-γ, and SB-AS2.

Several other types of Th1-type immunogens can be used (e.g., as an adjuvant) in compositions and methods of the present invention. These include, but are not limited to, the following. In some embodiments, monophosphoryl lipid A (e.g., in particular 3-de-O-acylated monophosphoryl lipid A (3D-MPL)), is used. 3D-MPL is a well known adjuvant manufactured by Ribi Immunochem, Montana. Chemically it is often supplied as a mixture of 3-de-O-acylated monophosphoryl lipid A with either 4, 5, or 6 acylated chains. In some embodiments, diphosphoryl lipid A, and 3-O-deacylated variants thereof are used. Each of these immunogens can be purified and prepared by methods described in GB 2122204B, hereby incorporated by reference in its entirety. Other purified and synthetic lipopolysaccharides have been described (See, e.g., U.S. Pat. No. 6,005,099 and EP 0 729 473; Hilgers et al., 1986, Int. Arch. Allergy. Immunol., 79(4):392-6; Hilgers et al., 1987, Immunology, 60(1):141-6; and EP 0 549 074, each of which is hereby incorporated by reference in its entirety). In some embodiments, 3D-MPL is used in the form of a particulate formulation (e.g., having a small particle size less than 0.2 □m in diameter, described in EP 0 689 454, hereby i0corporated by reference in its entirety). In some embodiments, saponins are used as an immunogen (e.g., Th1-type adjuvant) in a composition of the present invention. Saponins are well known adjuvants (See, e.g., Lacaille-Dubois and Wagner (1996) Phytomedicine vol 2 pp 363-386). Examples of saponins include Quil A (derived from the bark of the South American tree Quillaja Saponaria Molina), and fractions thereof (See, e.g., U.S. Pat. No. 5,057,540; Kensil, Crit Rev Ther Drug Carrier Syst, 1996, 12 (1-2):1-55; and EP 0 362 279, each of which is hereby incorporated by reference in its entirety). Also contemplated to be useful in the present invention are the haemolytic saponins QS7, QS17, and QS21 (HPLC purified fractions of Quil A; See, e.g., Kensil et al. (1991). J. Immunology 146,431-437, U.S. Pat. No. 5,057,540; WO 96/33739; WO 96/11711 and EP 0 362 279, each of which is hereby incorporated by reference in its entirety). Also contemplated to be useful are combinations of QS21 and polysorbate or cyclodextrin (See, e.g., WO 99/10008, hereby incorporated by reference in its entirety.

In some embodiments, an immunogenic oligonucleotide containing unmethylated CpG dinucleotides (“CpG”) is used as an adjuvant. CpG is an abbreviation for cytosine-guanosine dinucleotide motifs present in DNA. CpG is known in the art as being an adjuvant when administered by both systemic and mucosal routes (See, e.g., WO 96/02555, EP 468520, Davis et al., J. Immunol, 1998, 160(2):870-876; McCluskie and Davis, J. Immunol., 1998, 161(9):4463-6; and U.S. Pat. App. No. 20050238660, each of which is hereby incorporated by reference in its entirety). For example, in some embodiments, the immunostimulatory sequence is Purine-Purine-C-G-pyrimidine-pyrimidine; wherein the CG motif is not methylated.

Although an understanding of the mechanism is not necessary to practice the present invention and the present invention is not limited to any particular mechanism of action, in some embodiments, the presence of one or more CpG oligonucleotides activate various immune subsets including natural killer cells (which produce IFN-γ) and macrophages. In some embodiments, CpG oligonucleotides are formulated into a composition of the present invention for inducing an immune response. In some embodiments, a free solution of CpG is co-administered together with an antigen (e.g., present within a solution (See, e.g., WO 96/02555; hereby incorporated by reference). In some embodiments, a CpG oligonucleotide is covalently conjugated to an antigen (See, e.g., WO 98/16247, hereby incorporated by reference), or formulated with a carrier such as aluminium hydroxide (See, e.g., Brazolot-Millan et al., Proc. Natl. Acad Sci., USA, 1998, 95(26), 15553-8).

In some embodiments, adjuvants such as Complete Freunds Adjuvant and Incomplete Freunds Adjuvant, cytokines (e.g., interleukins (e.g., IL-2, IFN-γ, IL-4, etc.), macrophage colony stimulating factor, tumor necrosis factor, etc.), detoxified mutants of a bacterial ADP-ribosylating toxin such as a cholera toxin (CT), a pertussis toxin (PT), or an E. Coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (See, e.g., WO93/13202 and WO92/19265, each of which is hereby incorporated by reference), and other immunogenic substances (e.g., that enhance the effectiveness of a composition of the present invention) are used with a composition comprising an immunogen of the present invention.

Additional examples of adjuvants that find use in the present invention include poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

Adjuvants may be added to a composition comprising an immunogen, or, the adjuvant may be formulated with carriers, for example liposomes, or metallic salts (e.g., aluminium salts (e.g., aluminium hydroxide)) prior to combining with or co-administration with a composition.

In some embodiments, a composition comprising an immunogen comprises a single adjuvant. In other embodiments, a composition comprises two or more adjuvants (See, e.g., WO 94/00153; WO 95/17210; WO 96/33739; WO 98/56414; WO 99/12565; WO 99/11241; and WO 94/00153, each of which is hereby incorporated by reference in its entirety).

In some embodiments, a composition comprising an immunogen comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose. Although an understanding of the mechanism is not necessary to practice the present invention and the present invention is not limited to any particular mechanism of action, in some embodiments, use of a mucoadhesive (e.g., in a composition comprising an immunogen) enhances induction of an immune response in a subject (e.g., administered a composition of the present invention) due to an increase in duration and/or amount of exposure to an immunogen that a subject experiences when a mucoadhesive is used compared to the duration and/or amount of exposure to an immunogen in the absence of using the mucoadhesive.

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, subcutaneous, intramuscular, intraperitoneal, intravenous, or administration via other routes may be found in Remington's Pharmaceutical Sciences, Mack Publishing Company, Easton, Pa.

A composition comprising an immunogen of the present invention can be used therapeutically (e.g., to enhance an immune response) or as a prophylactic (e.g., for immunization (e.g., to prevent signs or symptoms of disease)). A composition comprising an immunogen of the present invention can be administered to a subject via a number of different delivery routes and methods.

For example, the compositions of the present invention can be administered to a subject (e.g., mucosally (e.g., nasal mucosa, vaginal mucosa, etc.)) by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal surface); being placed on or impregnated onto a nasal and/or vaginal applicator and applied; being applied by a controlled-release mechanism; being applied as a liposome; or being applied on a polymer.

In some embodiments, compositions of the present invention are administered mucosally (e.g., using standard techniques; See, e.g., Remington: The Science and Practice of Pharmacy, Mack Publishing Company, Easton, Pa., 19th edition, 1995 (e.g., for mucosal delivery techniques, including intranasal, pulmonary, vaginal and rectal techniques), as well as European Publication No. 517,565 and Ilium et al., J. Controlled Rel., 1994, 29:133-141 (e.g., for techniques of intranasal administration), each of which is hereby incorporated by reference in its entirety). Alternatively, the compositions of the present invention may be administered dermally or transdermally, using standard techniques (See, e.g., Remington: The Science arid Practice of Pharmacy, Mack Publishing Company, Easton, Pa., 19th edition, 1995). The present invention is not limited by the route of administration.

Although an understanding of the mechanism is not necessary to practice the present invention and the present invention is not limited to any particular mechanism of action, in some embodiments, mucosal vaccination is the preferred route of administration as it has been shown that mucosal administration of antigens has a greater efficacy of inducing protective immune responses at mucosal surfaces (e.g., mucosal immunity), the route of entry of many pathogens. In addition, mucosal vaccination, such as intranasal vaccination, may induce mucosal immunity not only in the nasal mucosa, but also in distant mucosal sites such as the genital mucosa (See, e.g., Mestecky, Journal of Clinical Immunology, 7:265-276, 1987). More advantageously, in further preferred embodiments, in addition to inducing mucosal immune responses, mucosal vaccination also induces systemic immunity. In some embodiments, non-parenteral administration (e.g., muscosal administration of vaccines) provides an efficient and convenient way to boost systemic immunity (e.g., induced by parenteral or mucosal vaccination (e.g., in cases where multiple boosts are used to sustain a vigorous systemic immunity)). In some embodiments, a composition comprising an immunogen of the present invention may be used to protect or treat a subject susceptible to, or suffering from, disease by means of administering a composition of the present invention via a mucosa route (e.g., an oral/alimentary or nasal route). Alternative mucosal routes include intravaginal and intra-rectal routes. In preferred embodiments of the present invention, a nasal route of administration is used, termed “intranasal administration” or “intranasal vaccination” herein. Methods of intranasal vaccination are well known in the art, including the administration of a droplet or spray form of the vaccine into the nasopharynx of a sujbect to be immunized. In some embodiments, a nebulized or aerosolized composition is provided. Enteric formulations such as gastro resistant capsules for oral administration, suppositories for rectal or vaginal administration also form part of this invention. Compositions of the present invention may also be administered via the oral route. Under these circumstances, a composition comprising an immunogen may comprise a pharmaceutically acceptable excipient and/or include alkaline buffers, or enteric capsules. Formulations for nasal delivery may include those with dextran or cyclodextran and saponin as an adjuvant.

Compositions of the present invention may also be administered via a vaginal route. In such cases, a composition comprising an immunogen may comprise pharmaceutically acceptable excipients and/or emulsifiers, polymers (e.g., CARBOPOL), and other known stabilizers of vaginal creams and suppositories. In some embodiments, compositions of the present invention are administered via a rectal route. In such cases, compositions may comprise excipients and/or waxes and polymers known in the art for forming rectal suppositories.

In some embodiments, the same route of administration (e.g., mucosal administration) is chosen for both a priming and boosting vaccination. In some embodiments, multiple routes of administration are utilized (e.g., at the same time, or, alternatively, sequentially) in order to stimulate an immune response.

For example, in some embodiments, a composition comprising an immunogen is administered to a mucosal surface of a subject in either a priming or boosting vaccination regime. Alternatively, in some embodiments, the composition is administered systemically in either a priming or boosting vaccination regime. In some embodiments, a composition comprising an immunogen is administered to a subject in a priming vaccination regimen via mucosal administration and a boosting regimen via systemic administration. In some embodiments, a composition comprising an immunogen is administered to a subject in a priming vaccination regimen via systemic administration and a boosting regimen via mucosal administration. Examples of systemic routes of administration include, but are not limited to, a parenteral, intramuscular, intradermal, transdermal, subcutaneous, intraperitoneal or intravenous administration. A composition comprising an immunogen may be used for both prophylactic and therapeutic purposes.

In some embodiments, compositions of the present invention are administered by pulmonary delivery. For example, a composition of the present invention can be delivered to the lungs of a subject (e.g., a human) via inhalation (e.g., thereby traversing across the lung epithelial lining to the blood stream (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989;84:1145-1146; Oswein, et al. “Aerosolization of Proteins”, 1990; Proceedings of Symposium on Respiratory Drug Delivery II Keystone, Colorado; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for dispensing of the therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

Thus, in some embodiments, a composition comprising an immunogen of the present invention may be used to protect and/or treat a subject susceptible to, or suffering from, a disease by means of administering the composition by mucosal, intramuscular, intraperitoneal, intradermal, transdermal, pulmonary, intravenous, subcutaneous or other route of administration described herein. Methods of systemic administration of the vaccine preparations may include conventional syringes and needles, or devices designed for ballistic delivery of solid vaccines (See, e.g., WO 99/27961, hereby incorporated by reference), or needleless pressure liquid jet device (See, e.g., U.S. Pat. No. 4,596,556; U.S. Pat. No. 5,993,412, each of which are hereby incorporated by reference), or transdermal patches (See, e.g., WO 97/48440; WO 98/28037, each of which are hereby incorporated by reference). The present invention may also be used to enhance the immunogenicity of antigens applied to the skin (transdermal or transcutaneous delivery, See, e.g., WO 98/20734 ; WO 98/28037, each of which are hereby incorporated by reference). Thus, in some embodiments, the present invention provides a delivery device for systemic administration, pre-filled with the vaccine composition of the present invention. The present invention is not limited by the type of subject administered (e.g., in order to stimulate an immune response (e.g., in order to generate protective immunity (e.g., mucosal and/or systemic immunity))) a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have been or are likely to become exposed to a microorganism (e.g., E. coli). In some embodiments, the human subjects are subjects that are more likely to receive a direct exposure to pathogenic microorganisms or that are more likely to display signs and symptoms of disease after exposure to a pathogen (e.g., immune suppressed subjects). In some embodiments, the general public is administered (e.g., vaccinated with) a composition of the present invention (e.g., to prevent the occurrence or spread of disease). For example, in some embodiments, compositions and methods of the present invention are utilized to vaccinate a group of people (e.g., a population of a region, city, state and/or country) for their own health (e.g., to prevent or treat disease). In some embodiments, the subjects are non-human mammals (e.g., pigs, cattle, goats, horses, sheep, or other livestock; or mice, rats, rabbits or other animal). In some embodiments, compositions and methods of the present invention are utilized in research settings (e.g., with research animals).

A composition of the present invention may be formulated for administration by any route, such as mucosal, oral, transdermal, intranasal, parenteral or other route described herein. The compositions may be in any one or more different forms including, but not limited to, tablets, capsules, powders, granules, lozenges, foams, creams or liquid preparations.

Topical formulations of the present invention may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxyethyl)pyrrolidone and a cell-envelope disordering compound, a sugar ester in combination with a sulfoxide or phosphine oxide, and sucrose rnonooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants. In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

In some embodiments, pharmaceutical compositions of the present invention may be formulated and used as foams. Pharmaceutical foams include formulations such as, but not limited to, emulsions, microemulsions, creams, jellies and liposomes. While basically similar in nature these formulations vary in the components and the consistency of the final product. The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the immunogen or other components of the formulation. In some embodiments, immunostimulatory compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, vaccine compositions are co-administered with one or more antibiotics. For example, one or more antibiotics may be administered with, before and/or after administration of the composition. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

There are an enormous amount of antimicrobial agents currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a vaccine composition comprising an immunogen with one or more additional active and/or immunostimulatory agents (e.g., a composition comprising a different immunogen, an antibiotic, anti-oxidant, etc.). Indeed, it is a further aspect of this invention to provide methods for enhancing prior art immunostimulatory methods (e.g., immunization methods) and/or pharmaceutical compositions by co-administering a composition of the present invention. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, adjuvants, etc.) can be any of the well-known agents in the art, including, but not limited to, those that are currently in clinical use.

In some embodiments, a composition comprising an immunogen is administered to a subject via more than one route. For example, a subject that would benefit from having a protective immune response (e.g., immunity) towards a pathogenic microorganism may benefit from receiving mucosal administration (e.g., nasal administration or other mucosal routes described herein) and, additionally, receiving one or more other routes of administration (e.g., parenteral or pulmonary administration (e.g., via a nebulizer, inhaler, or other methods described herein). In some preferred embodiments, administration via mucosal route is sufficient to induce both mucosal as well as systemic immunity towards an immunogen or organism from which the immunogen is derived. In other embodiments, administration via multiple routes serves to provide both mucosal and systemic immunity. Thus, although an understanding of the mechanism is not necessary to practice the present invention and the present invention is not limited to any particular mechanism of action, in some embodiments, it is contemplated that a subject administered a composition of the present invention via multiple routes of administration (e.g., immunization (e.g., mucosal as well as airway or parenteral administration of the composition) may have a stronger immune response to an immunogen than a subject administered a composition via just one route.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

In some embodiments, a vaccine composition of the present invention is formulated in a concentrated dose that can be diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of a nanemulsion and immunogen present in the concentrated composition. Concentrated compositions are contemplated to be useful in a setting in which large numbers of subjects may be administered a composition of the present invention (e.g., an immunization clinic, hospital, school, etc.). In some embodiments, a composition comprising an immunogen of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks. In some embodiments, following an initial administration of a composition of the present invention (e.g., an initial vaccination), a subject may receive one or more boost administrations (e.g., around 2 weeks, around 3 weeks, around 4 weeks, around 5 weeks, around 6 weeks, around 7 weeks, around 8 weeks, around 10 weeks, around 3 months, around 4 months, around 6 months, around 9 months, around 1 year, around 2 years, around 3 years, around 5 years, around 10 years) subsequent to a first, second, third, fourth, fifth, sixth, seventh, eights, ninth, tenth, and/or more than tenth administration. Although an understanding of the mechanism is not necessary to practice the present invention and the present invention is not limited to any particular mechanism of action, in some embodiments, reintroduction of an immunogen in a boost dose enables vigorous systemic immunity in a subject. The boost can be with the same formulation given for the primary immune response, or can be with a different formulation that contains the immunogen. The dosage regimen will also, at least in part, be determined by the need of the subject and be dependent on the judgment of a practitioner.

Dosage units may be proportionately increased or decreased based on several factors including, but not limited to, the weight, age, and health status of the subject. In addition, dosage units may be increased or decreased for subsequent administrations (e.g., boost administrations). It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions, immunogens, and other components and for screening for new components. Thus, it is not intended that the present invention be limited to any particular subject and/or application setting.

The present invention further provides kits comprising the vaccine compositions comprised herein. In some embodiments, the kit includes all of the components necessary, sufficient or useful for administering the vaccine. For example, in some embodiments, the kits comprise devices for administering the vaccine (e.g., needles or other injection devices), temperature control components (e.g., refrigeration or other cooling components), sanitation components (e.g., alcohol swabs for sanitizing the site of injection) and instructions for administering the vaccine.

### EXPERIMENTAL

The following examples are provided in order to demonstrate and further illustrate certain preferred embodiments and aspects of the present invention and are not to be construed as limiting the scope thereof.

### EXAMPLE 1

Genomic Islands of Uropathogenic E. coli

This Example describes the identification of E. coli genes associate with urinary tract infections.

### A. Materials and Methods

Bacterial strains and culture conditions. E. coli CFT073 was isolated from blood from a patient admitted to the University of Maryland Medical System for the treatment of acute pyelonephritis (Mobley et al., 1990. Infect. Immun. 58:1281-1289). This strain is highly virulent in the CBA/J mouse model of ascending UTI (Mobley et al., 1993. Mol. Microbiol. 10:143-155), is cytotoxic for cultured human renal proximal tubular epithelial cells (Mobley et al., 1990. Infect. Immun. 58:1281-1289), and has been sequenced and annotated (Welch et al., 2002. Proc. Natl. Acad. Sci. USA 99:17020-17024).

For growth on solid medium, bacterial strains were streaked onto LB agar plates (10 g tryptone, 5 g yeast extract, 10 g NaCI, 15 g agar [all per liter]) and incubated at 37° C. for 18 h. For growth in liquid culture, strains were inoculated into LB broth (10 g tryptone, 5 g yeast extract, 10 g NaCl [all per liter]) and incubated at 37° C. for 18 h with aeration (200 rpm). Kanamycin (25 μg/ml), chloramphenicol (20 μg/ml), or ampicillin (100 μg/ml) was added as appropriate.

Strains to be tested in the mouse model of ascending UTI were cultured overnight in the absence of antibiotic selection (with the exception of strains containing a vector for in vivo complementation), resuspended in phosphate-buffered saline (PBS), and adjusted to 4.0×10⁹ CFU/ml.

For growth on CAS agar (Schwyn et al., 1987. Anal. Biochem. 160:47-56) containing 2 mM FeCl₃, bacterial strains were cultured overnight in LB broth and standardized in PBS to 1×10⁹ CFU/ml. A sample (20 μl) of the suspension was spotted onto CAS agar plates, and the plates were incubated at room temperature overnight. Siderophore production was indicated by orange halos around the bacterial growth. The enterobactin/aerobactin (entF::kan iucB::cam) siderophore-deficient strain of CFT073 (Torres et al., 2001. Infect. Immun. 69:6179-6185) was kindly provided by Alfredo Torres, Department of Microbiology and Immunology, University of Texas Medical Branch, Galveston. For growth under iron-limiting conditions, strains were cultured overnight in LB broth containing 200 μM 2,2′-dipyridyl and washed twice in PBS. LB broth or M9 minimal medium containing 200, 300, 400, 500, or 600 μM 2,2′-dipyridyl was inoculated (in triplicate) with 1×10⁸ CFU/ml of bacterial suspension. Each well contained 300 μl of medium, corresponding to 3×10⁷ CFU/well. Growth curves were performed using the Bioscreen growth curve analyzer (Growth Curves USA) with growth at 37° C. with continuous shaking.

Identification of genomic islands of E. coli CFT073. GIs of E. coli CFT073 were identified and defined using comparative genomic hybridization microarray analysis of seven UPEC strains and three fecal/commensal E. coli strains as described previously (Lloyd et al., 2007. J. Bacteriol. 189:3532-3546). The presence of several of these GIs was demonstrated by sequence comparison with E. coli 536 (Brzuszkiewicz et al., 2006. Proc. Natl. Acad. Sci. USA 103:12879-12884).

Construction of genomic island mutants. Isogenic mutants in E. coli CFT073 were constructed using the lambda red recombinase system (Datsenko and Wanner. 2000. Proc. Natl. Acad. Sci. USA 97:6640-6645). Briefly, primers homologous to sequences within the 5′ and 3′ ends of the target regions were designed (H1 and H2 primers, respectively; Table 1) and were used to replace these genes with a nonpolar kanamycin or chloramphenicol resistance cassette derived from plasmid pKD4 or pKD3, respectively (Datsenko and Wanner. 2000, supra). Less than 10% of the targeted gene sequence (with the exception of Δc0363, at 13.4%) remained after homologous recombination. Kanamycin or chloramphenicol was used for selection of all deletion constructs. GI deletions ranged from 32 kb to 123 kb, and individual gene and potential operon deletions were also constructed using this method. The boundaries of each of the 11 GI mutants are defined in Table 2.

Genotypic analysis of mutants. To verify whether the kanamycin or chloramphenicol resistance cassette recombined within the target gene site, primers that flank target GI sequence were designed (Table 1). Both wild-type (where possible) and mutant gene sequences were amplified with genomic confirmation primers by PCR using Taq DNA polymerase (New England Biolabs). Additional confirmation that the desired genomic replacements had occurred was obtained by digesting each confirmation PCR product with the restriction enzyme EagI (New England Biolabs) for mutants containing a kanamycin resistance cassette or with EaeI (New England Biolabs) for mutants containing a chloramphenicol resistance cassette. The kanamycin resistance cassette contains a single EagI restriction site, and the chloramphenicol resistance cassette contains a single EaeI restriction site, confirming replacement of the GI with the antibiotic resistance cassette if bands of the predicted size were observed. PCR products and restriction enzyme digests were electrophoresed on a 1% agarose gel for visualization of the amplified and digested DNA. The 1-kb+DNA ladder (Invitrogen) was used to estimate DNA fragment size.

Growth rates of genomic island mutants. All mutants were tested for their ability to grow in of LB broth (10 g/liter NaCl) or sterile, pooled human urine using the Bioscreen growth curve analyzer (Growth Curves USA) to determine whether deletion mutants showed a growth defect in vitro. Growth rates of all deletion mutants were measured prior to in vivo testing of the isogenic mutants in the murine model of ascending UTI.

Murine model of ascending UTI. An adaptation (Johnson et al., 1987. J. Urol. 138:632-635) of the CBA/J mouse model of ascending UTI, originally developed by Hagberg et al. (1983. Infect. Immun. 40:273-283.), was used to assess virulence of E. coli CFT073 and its deletion constructs. Female 6- to 8-week-old CBA/J mice (Harlan Sprague-Dawley [Indianapolis, Ind.] or Jackson Laboratory [Bar Harbor, Me.]) were anesthetized with 100 mg ketamine and 10 mg xylazine per kg body weight and inoculated transurethrally with a 50 μl bacterial suspension delivering 2×10⁸ CFU per mouse. A sterile polyethylene catheter (inner diameter, 0.28 mm; outer diameter, 0.61 mm) connected to an infusion pump (Harvard Apparatus) was used to deliver the inoculum over a 30-second period. For independent challenges, mice were transurethrally inoculated with 2×10⁸ CFU of a single strain per mouse as described above. For cochallenge studies, two cultures were prepared as described above, mixed in a 1:1 ratio, and used to deliver a total of 2×10⁸ CFU per mouse. The two strains consisted of either wild-type CFT073 and an isogenic mutant or two different isogenic mutants. To determine the input CFU/ml for each strain, dilutions of each inoculum were plated on LB agar plates containing an antibiotic where required using an Autoplate 4000 (Spiral Biotech). Bacterial counts were determined using a Q-Count machine and accompanying software (Spiral Biotech).

Mice were sacrificed at 48 h postinoculation (hpi); the bladder and kidneys were aseptically removed, weighed, and homogenized in 3 ml sterile PBS. Homogenized tissue samples were plated onto LB plates with or without antibiotic (as required) to determine the output CFU/g of tissue for each strain. Wild-type bacterial counts were obtained by subtracting the CFU/ml of the antibiotic containing plates from the CFU/ml of the plain LB plates. The lower limit of detection of this assay is 10² CFU/g of tissue. Thus, for statistical analysis, this value (10² CFU/g of tissue) was assigned to samples with an undetectable level of colonization. All animal protocols were approved by the University Committee on Use and Care of Animals at the University of Michigan Medical School.

Cloning of the fbp locus into pGEN-MCS. One of the ferric binding protein (fbp) loci (c0294 to c0297 [c0294-97]) was PCR amplified using primers 5′NNNNNNTACGTAATGAAAACTCAAATAACTTTCGCTGCG-3′ (SEQ ID NO:1) (to introduce a 5′ SnaBI site) and 5′-GAGCTCCTATTTTTCGATTGCACCATC-C-3′ (SEQ ID NO:2) (to introduce a 3′ SacI site) and cloned into the pGEN-MCS vector (Lane et al., 2007. Proc. Natl. Acad. Sci. USA 104:16669-16674) under the control of the em7 promoter. The pGEN-luxCDABE (Pem7) (Lane et al., 2007. Proc. Natl. Acad. Sci. USA 104:16669-16674) construct, containing the luciferase (lux) genes under the control of the em7 promoter, was digested with SnaBI and SacI to replace the lux cassette with the c0294-97 genes. Total membrane preparations were isolated using a modified version of the protocol of Hagan and Mobley (Hagan and Mobley. 2009. Mol. Microbiol. 71:79-91) to visualize expression of the fbp locus. Briefly, 50-m1 overnight E. coli TOP10/pGEN and TOP10/pGENfbp cultures were pelleted by centrifugation (8,000 μg, 10 min, 4° C.), resuspended in 7 ml of 10 mM HEPES, and treated with 10 μl benzonase nuclease (Sigma; diluted to 10 U/μl in 50% glycerol, 20 mM Tris [pH 8], 2 mM MgCl2, and 20 mM NaCI). Cells were lysed by two passages through a French pressure cell (20,000 lb/in²), and total membrane was isolated from the cleared lysate by ultracentrifugation (100,000 μg, 30 min, 4° C.). Total membrane preparations were resuspended in 100 μl of 10 mM HEPES, solubilized in sodium dodecyl sulfatepolyacrylamide gel electrophoresis sample buffer, and electrophoresed on a 10% polyacrylamide gel.

Statistical analysis. Median CFU/g tissue was reported for all independent and cochallenge experiments to reflect the nonparametric distribution of the in vivo data. Statistically significant differences in colonization, defined as a P value of <0.05, were determined using InStat software (Graphpad, San Diego, Calif.). Independent challenges were analyzed using the unpaired, nonparametric Mann-Whitney test, and cochallenge infections were analyzed using the nonparametric Wilcoxon matched-pair test.

### B. Results

Isogenic mutants lacking PAI-CFT073-aspV, PAI-CFT073-metV, and PAI-CFT073-asnT are outcompeted by wild-type strain CFT073 in vivo. Eleven genomic islands (FIG. 1A), ranging from 32 kb to 123 kb, were individually deleted from E. coli CFT073 using the lambda red recombinase system. Deletions were verified by PCR analysis (FIG. 1B). In each case, PCR analysis confirmed that PAI sequences were absent and that flanking PAI boundaries were now close enough to each other to allow PCR amplification of the intervening sequence. The growth rates of the 11 GI mutants and wild-type CFT073 in LB broth or sterile, pooled human urine were comparable. However, the PAI-metV mutant entered stationary phase earlier than did strains with the other constructs when the strains were grown in LB broth), and the PAI-aspV mutant demonstrated a slight lag after 2 h of growth in human urine but had reached a similar A600 after 8 h of growth.

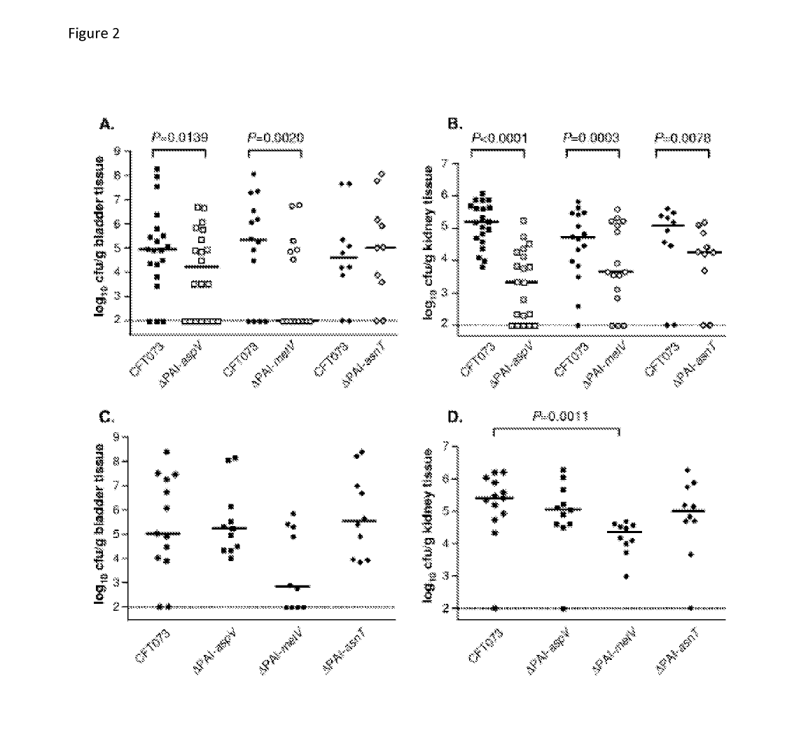

Nine isogenic GI mutants were tested for the ability to colonize the CBA/J mouse model of ascending UTI at 48 h after transurethral inoculation (Table 3). Two islands (PAICFT073-pheV and PAI-CFT073-pheU) were not considered further. Three mutants, ΔPAI-CFT073-aspV, ΔPAI-CFT073-metV, and ΔPAI-CFT073-asnT (subsequently referred to as ΔPAIaspV, ΔPAI-metV, and ΔPAI-asnT, respectively) were significantly outcompeted in vivo by wild-type strain CFT073. The ΔPAI-aspV and ΔPAI-metV mutants were significantly outcompeted by strain CFT073 in the bladders (FIG. 2A; P=0.0139 and P=0.0020, respectively) and in the kidneys (FIG. 2B; P<0.0001 and P=0.0003, respectively), while the ΔPAIasnT mutant was outcompeted in the kidneys (FIG. 2B; P=0.0078), indicating that these islands contribute to the fitness of CFT073 in vivo.

GI mutants ΔPAI-aspV, ΔPAI-met V, and ΔPAI-asnT were also assessed in independent challenges in the CBA/J mouse model of ascending UTI to determine whether these PAIs were required for colonization. Levels of colonization (CFU/g tissue) for ΔPAI-aspV and ΔPAI-asnT mutants were not statistically significantly different from that of wild-type strain CFT073 in the bladder (FIG. 2C) or kidneys (FIG. 2D). The ΔPAI-metV mutant colonized the kidneys (FIG. 2D) at a significantly lower level than that of strain CFT073 (P=0.0011) and tended to show attenuated colonization in the bladder (P=0.0668) (FIG. 2C). These data indicate-that genes present on PAI-aspV and PAI-asnT contribute to the fitness of UPEC in vivo, while a gene(s) on PAI-metV is required for colonization of the kidneys and possibly the bladder.

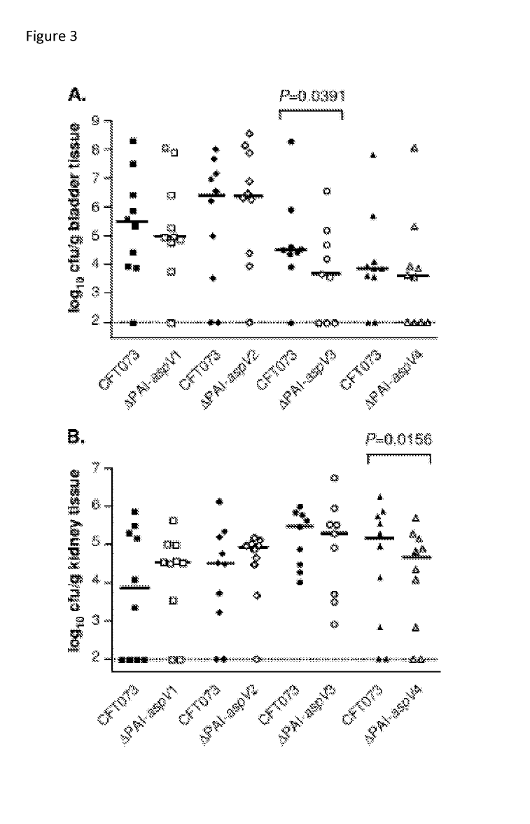

PAI-aspV region. The aspV GI is 99.7 kb in length, and its deletion attenuated the ability of UPEC strain CFT073 to colonize. To localize the observed phenotype of the ΔPAIaspV mutant to a specific region, four smaller deletion mutants, ΔPAI-aspVI (25.0 kb), ΔPAI-aspV2 (26.4 kb), ΔPAI-aspV3 (30.0 kb), and ΔPAI-aspV4 (17.4 kb), were constructed to cover the entire genomic island (FIG. 1A). Each of the four isogenic deletion mutants was tested in vivo by cochallenge with wild-type CFT073. The ΔPAI-aspV3 mutant was significantly outcompeted by wild-type strain CFT073 in the bladders of mice (P=0.0391) (FIG. 3A), while the ΔPAI-aspV4 mutant was significantly outcompeted in the kidneys (P=0.0156) (FIG. 3B).

Several genes with known or predicted roles in the pathogenicity of UPEC reside within the aspV GI, including the contact-dependent inhibition gene cdiA (Aoki et al., 2005. Science 309:1245-1248) and the autotransporter gene picU (Heimer et al., 2004. Infect. Immun. 72:593-597). One gene of interest, c0363, appears to be part of an operon consisting of open reading frames (ORFs) c0360 to c0363 (c0360-c0363). c0363 is annotated as putative RTX family exoprotein A gene, and on the basis of results of in silico analysis (Parham et al., 2005. J. Clin. Microbiol. 43:2425-2434), the c0360-63 locus has been reported to encode a type one secretion (tos) system, with c0363 designated as tosA. Additionally, the UPEC-specific operon (c0294-97), identified in a comparative genomic hybridization study of strain CFT073 (Lloyd et al., 2007. J. Bacteriol. 189:3532-3546), is present in the aspV pathogenicity island. Two identical copies of the c0294-97 operon exist in strain CFT073, one of which is located in PAI-aspV and the second in GI-CFT073-cobU(c2518-15). The PAI-aspV2 island contains c0294-97, PAI-aspV3 island contains cdiA (c0345) and picU (c0350), and PAI-aspV4 contains c0363. Isogenic mutants were constructed for cdiA (Δc0345), picU (Δc0350), one copy of the iron acquisition operon (Δc0294-97), and the type one secretion system gene tosA (Δc0363).

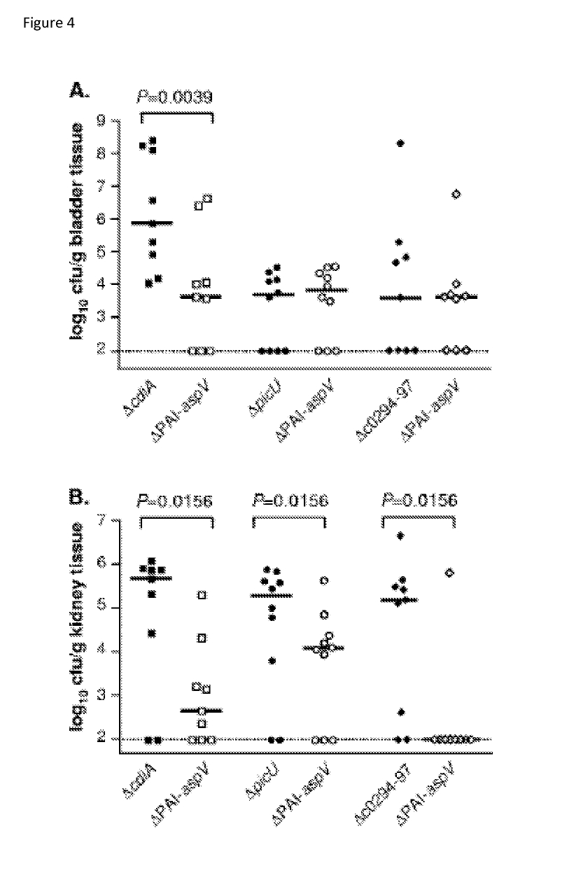

The ΔcdiA, ΔpicU and Δc0294-97 mutants were tested in cochallenge with the ΔPAI-aspV mutant (FIG. 4) to identify the contribution of these known or putative virulence genes to the ΔPAI-aspV phenotype. The ΔPAI-aspV mutant was significantly outcompeted by ΔcdiA in the bladder (P=0.0039) (FIG. 4A) and kidneys (P=0.0156) (FIG. 4B). There was no significant difference in the levels of colonization between ΔpicU and Δc0294-97 and ΔPAI-aspV mutants in the bladders of mice (FIG. 4A), indicating that picU and c0294-97 contribute to the ΔPAI-aspV phenotype in the bladder. However, the ΔPAIaspV mutant was outcompeted by ΔpicU and Δc0294-97 mutants in the kidneys (P=0.0156 and P=0.0156, respectively) (FIG. 4B).

A Δc0363 mutant was tested in cochallenge with wild-type CFT073 (FIG. 5). Deletion of c0363 resulted in statistically significant outcompetition of the Δc0363 mutant by strain CFT073 in both the bladder (P=0.0010) and kidneys (P=0.0002). To confirm this observation and minimize the possibility that a secondary mutation contributed to the attenuation of colonization, a second Δc0363 mutant (Δc0363 2, constructed independently using different primers) was tested in cochallenge with CFT073, also resulting in statistically significant outcompetition of the mutant by CFT073 in both the bladder (P=0.0156) and kidneys (P=0.0078). These data indicate that c0363 contributes to the fitness of CFT073 in the urinary tract.

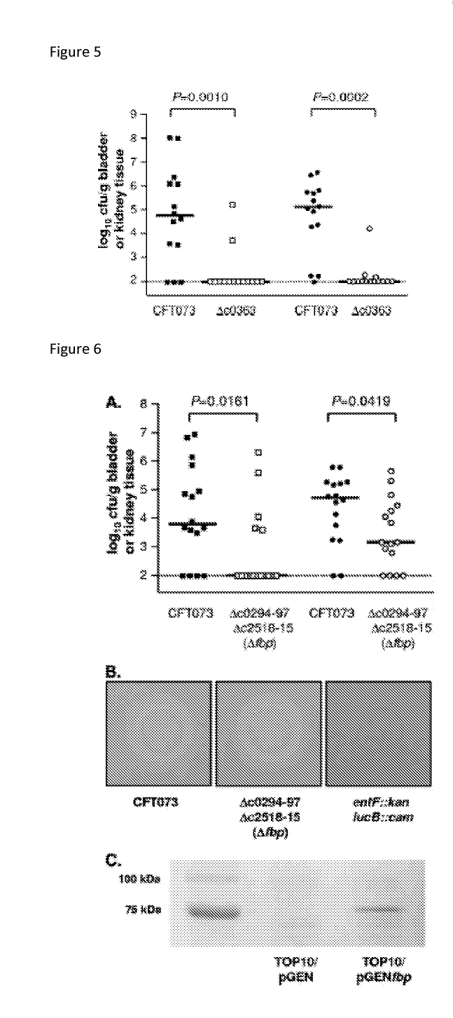

A mutant in which both copies of the putative iron acquisition system (Δc0294-97 Δc2518-15) had been deleted was tested in cochallenge with wild-type strain CFT073. The Δc0294-97 Δc2518-15 mutant was significantly attenuated in both the bladder (P=0.0161) and kidneys (P=0.0419) (FIG. 6A) of mice during cochallenge with strain CFT073. Wild-type CFT073, the Δc0294-97 Δc2518-15 mutant, and a siderophore deficient strain of CFT073 (entF::kan iucB::cam) were grown on CAS siderophore agar. No difference in halo formation was observed between wild-type CFT073 and the Δc0294-97 Δc2518-15 mutant (FIG. 6B), indicating that both strains are producing siderophores. The fbp locus (c0294-97) was cloned into pGEN-MCS under the control of the constitutive em7 promoter, and protein expression was confirmed examining whole-membrane preparations of E. coli TOP10/pGEN and TOP/pGENJbp (FIG. 6C). In order to restore the wild-type phenotype of the jbp (Δc0294-97 Δc2518-15) double mutant, in vivo complementation was attempted using cochallenge of CFT073/pGEN with Δfbp/pGENfbp. However, CFT073/pGEN significantly outcompeted Δfbp/pGENfbp in the kidneys (P=0.0156), while median levels of bladder colonization were at the limit of detection for both strains (P>0.9999). No difference in growth was observed between wild-type CFT073 and the Δc0294-97 Δc2518-15 double mutant under iron limiting conditions in LB broth or M9 minimal medium containing 200, 300, 400, 500, or 600 μM 2,2′-dipyridyl.

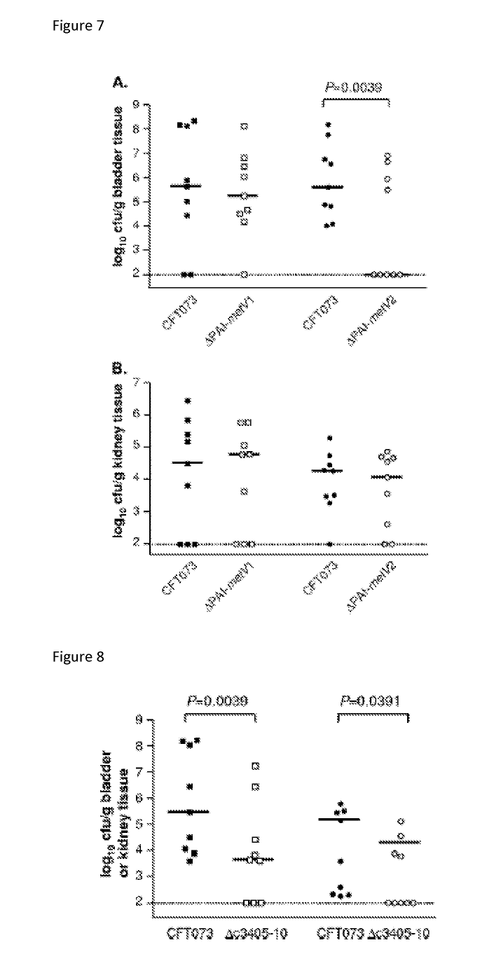

PAI-metV region. Since deletion of PAI-metV attenuated colonization in strain CFT073, two smaller mutants in PAImetV were constructed to examine this 32.1-kb GI in greater detail (FIG. 1A). Wild-type CFT073 was used in separate cochallenge experiments with strains carrying ΔPAI-metV1 (16.1 kb) and ΔPAI-met V2 (15.8 kb) (FIG. 7). The ΔPAI-metV2 mutant was significantly outcompeted by CFT073 in the bladder (P=0.0039) (FIG. 7A), with the median level of colonization of the deletion mutant at the limit of detection. In the bladder, the ΔPAI-metV2 mutant showed the same phenotype as the ΔPAI-metV whole-GI knockout, with both mutants showing a 3-log-unit decrease in colonization levels from the colonization level of CFT073. There was no significant difference in the levels of colonization between CFT073 and ΔPAI-metV1 mutant in the bladder (FIG. 7A), and the median CFU/g kidney tissue for the ΔPAI-metV1 or ΔPAI-metV2 mutant was not statistically significantly different from that of wild-type CFT073 (FIG. 7B).

The only two genes in PAI-metV with a predicted role in virulence encode the secreted protein Hcp (c3391) and the ClpB protein (c3392). Cochallenge of the ΔPAI-metV mutant with the Δc3391-92 mutant revealed that the ΔPAI-metV mutant colonized the bladder to significantly lower levels (P=0.0156) than the Δc3391-92 mutant did. A similar trend was seen in the kidneys of mice, although this difference was not statistically significant (P=0.0781).

The ΔPAI-metV2 mutant, which showed a 3-log-unit drop in colonization of the bladder from the colonization level of strain CFT073, was further characterized by constructing a strain with mutations in genes c3398-c3404, representing the first half of the genes in PAI-metV2. In cochallenge with CFT073, the Δc3398-c3404 mutant showed similar levels of colonization to the wild-type strain, and these differences were not statistically significant. The remaining genes in the PAI-metV2 region, c3405-10, were deleted and tested in cochallenge with CFT073. The ability of the Δc3405-10 deletion mutant to colonize was significantly attenuated in both the bladder (P=0.0039) and kidneys (P=0.0391) than in wild-type CFT073, as shown in (FIG. 8).

TABLE 1
Primers used in this study

| | | | Primers used to confirm | |
|---|---|---|---|---|
| | Primers used to construct | | isogenic mutants | |
| | isogenic mutants | | Primer | |
| | Primer | | direc- | |
| Mutant | type^{a} | Primer sequence (5′-3′) | tion^{b} | Primer sequence (5′-3′) |
| ΔPAI-CFT073-pheV | H1 | ACACAGCGATAAAGTACTCAAAAGCCTCGAGACTCACG | Fwd | CAGTCGGTAGAGCAGGGGATTGAA |
| | H2 | TATTGCCATTTCCTTAACCCCACCTGATAACCCTTAGC | Rev | AGCGACTGGAGTTTGGGCGGGGGTAGG |
| ΔPAI-CFT073-pheU | H1 | CAGGCTGATGGTACATGCTCTGAAACTGGCTGCAGGATACG | Fwd | GCACAGAAGGAAAGTACCTGGCTATTA |
| | H2 | TCGCTTTTACTGAAATTAGGTTGACGAGATGTGCAGATTACG | Rev | GGAGATGGTTGCTGAACGTGTGGATTA |
| ΔPAI-CFT073-aspV | H1 | GTGCAGTTCCCTTCTGAAAATACTTAATCACAAACATCTCA | Fwd | TCAGACAACTCACTCACCTCTCATCTC |
| | H2 | ATAACCCATCAGCCCGCTTCTGTAATACCTCCATTCGTTCTA | Rev | TGAAATTATACTGAACGGATACAAGAC |
| Δφ-CFT073-b0847 | H1 | GGGCCTCTATCTTCAATCTGTTCGACTAACCCCTCCTCT | Fwd | CTTTGTCGCCACTTGTTTTACCTTAGA |
| | H2 | AGAATCATTCCATTTCGAAATCATTAATCTTCACTTCAAG | Rev | CGATGGCGACAAATTGGCGGCAGAGTC |
| ΔPAI-CFT073-serX | H1 | GTATTGCTGAAGCTGCACGTACTGCCCGGATAATGCGAGAG | Fwd | GTAAAGGGGCGGGGGAAATGGGTTTTT |
| | H2 | CTTTTCCCTGAAGAGACCGGATGTGATCGTCCAGATGAATAG | Rev | ACAGGAACAACAATTTGGTGAGGTGTC |
| Aφ-CFT073-potB | H1 | CTTCTTTTAACGTTATCCCAATGGCATCACAGCGTAGTGTAA | Fwd | TTATAGCGATCGGTGGTTGCCTGGACT |
| | H2 | GGCTTCTTCCAGTGGTACGTAATTTTCTCCGTTTCCCGATAC | Rev | ACGTGGAACAAATAGACACAAGAAATA |
| ΔPAI-CFT073-asnT | H1 | CCTTACCGACGCAAAAATCCGCACCCTCAAGCCTTCTGATAA | Fwd | GCCCCGTTCTCACGATTCCTCTGTAGT |
| | H2 | CAGCGTGATTCTTGCGGTACCGAAGCGGCTTAACCAGTCTGT | Rev | GCATTCGTGACGTTCGGCACATAGTTC |
| ΔGI-CFT073-asnW | H1 | ACCCCCATATGTCCCTTAACGACGCAAAAATCCGTAGTCTCA | Fwd | GCATCGCTAATATTCGCCTCGTTCTCA |
| | H2 | GGAAGCGCTGATCCTCTCCCCTAGTGGAACTGTGTCTAAAGZ | Rev | CGGGAATGCCTGTGCAAATTAGTTCTG |
| ΔGI-CFT073-cobU | H1 | GGTTGACCTAAGGTAGCAGTTTATCCTGATGCGCTGAGATTT | Fwd | GCTGACATCATCAAGAATAAAAAGGTT |
| | H2 | CACGGAAACAGAAGGTGTGGTGGAATTATGCGAAGAGGTT | Rev | TTCCGGATGTTGCAGGGCCTTAAT |
| ΔPAI-CFT073-metV | H1 | AGTAAACCGTTAATATCCCTCCATCAAAGCCATCCATCTTAT | Fwd | TTTTTCGTTTTTACGCTTCCTTAC |
| | H2 | TTGTAACTGTTAAATCAGGCAAGGCAATGTTTGAAGTAGT | Rev | TATTAGACGCTGGTTTTGTGACTGATG |
| ΔGI-CPT073-selC | H1 | AAAAACTGATCTGGGGGATGTAGAAACTCAAGGAAGTAG | Fwd | TCCTTGATGCTATAGGGGTGCTGAGAC |
| | H2 | ATGACGGTGAGGGAGTAGAGTAATCAATCAGTTTTAGTGAAT | Rev | ACCCATTTTTCCCTCTGCATACTGTTT |
| ΔPAI-CFT073-aspV1 | H1 | GGAGCGGTAGTTCAGTCGGTTAGAATACCTGCCTGTCA | Fad | AACAGCAACAAGGTGAAACAACAAT |
| | H2 | CGAAAAATCACTAACGAAACATTGGATCCCCATTGTTGC | Rev | CTGAGAGCGAGGAGCGGAAGTAAG |
| ΔPAI-CFT073-aspV2 | H1 | CAGCTTTCTGACAACCCGGCCGCTCCTGCTTTCAACAA | Fwd | TACATGCTTACTTCCGCTCCTCGCTCTC |
| | H2 | GGTTTTTAATGCACACTGGATAACAACCGGACGAATCT | Rev | TAGTCTGATATGGCTTTTGTCGCTGTCC |
| ΔPAI-CFT073-aspV3 | H1 | TGATAAGCCAAATTGATAAGCTGGAATATGTGATGAAAGTGC | Fwd | CAGCGCCAGTGATATTTGAAGATTCGTC |
| | H2 | CTTGTCATATCCGGATAAAATCACCCTCTGGTAATACTCTTA | Rev | TGCACATCGCACAAGTGATTATGAACAG |
| ΔPAI-CFT073-aspV4 | H1 | GAATTAAGCGCCAGACGTATGGTCAAAAGTAGTGGAGTAGAA | Fwd | TTATTGTTACCTTCTTTTGTTGTGATGA |
| | H2 | CATGGCGGGATGCGGATGAGTTTAGGTTGCTGTGAGTG | Rev | TTGATGCGGAGTTGTCGATGGCTGTATT |
| ΔcdiA (c0345) | H1 | AGCCTCCCGTTCGCTTCACTTACCGCCTGCTGAGTTAC | Fwd | TTCACTGCCGGACTGCCTCTGGTT |
| | H2 | CCGTAGAAAGCCCCACTGCACCAACGCCAAAACCACCATTTA | Rev | AGCGCAAGCATCAATAAAAATAGT |
| ΔpicU (c350) | H1 | GGGACTTATTGTTGTCTCTGAACTTGCCAGCAGGGTA | Fwd | ACATCATGGAGAGTCCGCAGTGAA |
| | H2 | CGCATCACGCAGTACCGTCTCACCATTATTCAGTAGG | Rev | GCTGACTTCTAAACTCCAGACCA |
| Δc0294-97 | H1 | GCTCCTCGCTCTCAGCGGACCTTCAGCTCAGTGATATCG | Fwd | GGGGATCCAATGTTTCGTTAGTGA |
| (fbpABCD) | H2 | CCTGAGGCTGAAGCAACCACGTTAATCCGACTATTTTTCG | Rev | TGGTAGGCGGATAGATAATAGAAA |
| Δc0363 #1^{c} | H1 | CTGTCGGAGGTCGTCATACAGTTAAAGCACAG | Fwd | TACCGATGGTGATGGTGCAACAGA |
| | H2 | GTTAATATAGCCCTGATTACCGGAGGAGGTGGAGTC | Rev | ATGGATACTTTACCGGCAGCCACT |
| Δc0363 #2 | H1 | AGGAAGAAGATACTGAGGTCCGGATAGAGGGATTCTGG | Fwd | TGTCGGAGGTCGTCATACAGTT |
| | H2 | CCGTTAATATAGCCCTGATTACCGGAGGAGGTGGAGTC | Rev | TAACAGTTGTTGCCGTTGCCGT |
| ΔPAI-CFT073-metV1 | H1 | AAGGATATGGCCGACAGTTTCCAGAATGAAGTTCCCGC | Fwd | ATTCTCTCACCAGATAATGCCGCC |
| | H2 | TATTCCTTCTGGGCGCGAACGATAGCCTGTATAAAGCG | Rev | ATCAACAGACGAAGCCAGACAGCA |
| ΔPAI-CFT073-metV2 | H1 | CTTTTGGGTGTGGGGTTACTGCTCCTTGTTGTGTTGTTGC | Fwd | CATTGCCAGGCATCGTCTTTGGTT |
| | H2 | CAATGAATTTATATTTCGTTGAATAGATAACATTTACC | Rev | ACAGGCTGGGTTTGCCGTACTAAA |
| Δc3391-92 | H1 | AGAAGGCGGTGCTTCAATCACACTAACAAGGAGAGTAA | Fwd | CAGTCAACCGGCGTGTCGAAATCAGTCT |
| (Hcp, ClpB) | H2 | GTGGTGTGCGTGGTCGAAGAACTGTACGTTCATAAGAG | Rev | CTCGCGGCCTTCAAAGGTCAGCACATCC |
| Δc3398-c3404 | H1 | GCAGAACGAAGCCTCGACGATATCACTATACGCTCAACC | Fwd | TGGGGTTACTGCTCCTTGTTGTG |
| | H2 | CTGATTTAACCGGGTATCAATTTGCGTCAACAGCGTTGGC | Rev | GCGGTTTGTCAGCATTCTAA |
| Δc3405-c3410 | H1 | TTTTGTGACTGATGTCGGATATTTGAATGTCGGCTTG | Fwd | GACGCTGGCGAGAAGGGGATAA |
| | H2 | ATCTCCCTTCCTGCGAAGTAATCAATTATCGACTGGG | Rev | ATTTCGGTAGATAGCTTGGGTTCG |

^{a}Primers homologous to sequences within the 5′ and 3′ ends of the target regions were designed (H1 and H2 primers, respectively) and were used to replace these genes with a nonpolar kanamycin or chloramphenicol resistance cassette derived from plasmid pKD4 or pkD3, respectively (12).
^{b}Forward (Fwd) and reverse (Rev) genomic primers.
^{c}Two independently constructed Δc0363 mutants were tested in this study. Δc0363 #1 is referred to as Δc0363 throughout the article.

TABLE 2
TABLE 2. Characteristics of genomic and pathogenicity island deletion constructs in E. coli CFT073

| | Size | CDS^{a} in | | tRNA gene present |
|---|---|---|---|---|
| Genomic island | (kb) | island | Region replaced in λ red mutant | in mutant?^{b} |
| PAI-CFT073-pheV (PAI I) | 123 | c3556-kpsM | Nucleotide 383 of c3556 to 1134 nucleotides | Yes (pheV) |
| | | | downstream of kpsM (intergenic region) | |
| PAI-CFT073-pheU (PAI II) | 52 | c5143-c5216 | Nucleotide 1319 of c5144 to 290 nucleotide | No (pheU) |
| | | | upstream of c5216 | |
| PAI-CFT073-aspV (PAI III) | 100 | c0253-c0368 | Nucleotide 221 of c0253 to 9 nucleotides | Yes (aspV) |
| | | | upstream of c0368 | |
| φ-CFT073-b0847 | 33 | inT-ogrK | 206 nucleotides downstream of ybjK to 94 | NA |
| | | | nucleotides upstream of ogrK | |
| PAI-CFT073-serX | 113 | c1165-c1293 | Nucleotide 232 of c1165 to nucleotide 664 | Yes (serX) |
| | | | of c1292 | |
| φ-CFT073-potB^{c} | 44 | c1400-c1474 | Nucleotide 772 of c1400 to nucleotide 242 | NA |
| | | | of c1506 | |
| PAI-CFT073-asnT (HPI) | 32 | c2418-c2437 | 172 nucleotides upstream of c2418 to | Yes (asnT) |
| | | | nucleotide 657 of c2437 | |
| GI-CFT073-asnW | 54 | c2449-c2475 | Nucleotide 35 of c2449 lo 23 nucleotides | Yes (asnW) |
| | | | upstream ofc2475 | |
| GI-CFT073-cobU | 44 | c2482-c2528 | 969 nucleotides upstream of c2482 to 50 | NA |
| | | | nucleotides downstream of c2528 | |
| PAI-CFT073-metV | 32 | c3385-c3410 | 475 nucleotides upstream of c3385 to 53 | Yes (metV) |
| | | | nucleotides upstream of c3410 | |
| GI-CFT073-selC | 68 | intC-c4581 | 432 nucleotides upstream of intT to 65 | No (selC) |
| | | | nucleotides downstream of c4581 | |

^{a}CDS, coding sequence.
^{b}PAIs are commonly inserted adjacent to tRNA genes: deletion of individual GIs generally did not disrupt the adjacent tRNA gene. NA, not applicable (GI is not located near a tRNA gene).
^{c}PAI-CFT073-potB was originally designated as c1400-c1507, so this region was replaced in the GI isogenic mutant. However, the boundaries of the island have since been redefined as c1400-c1474, since c1475-c1507 was a separate island (<30 kb), so it was excluded from our analysis.

### EXAMPLE 2

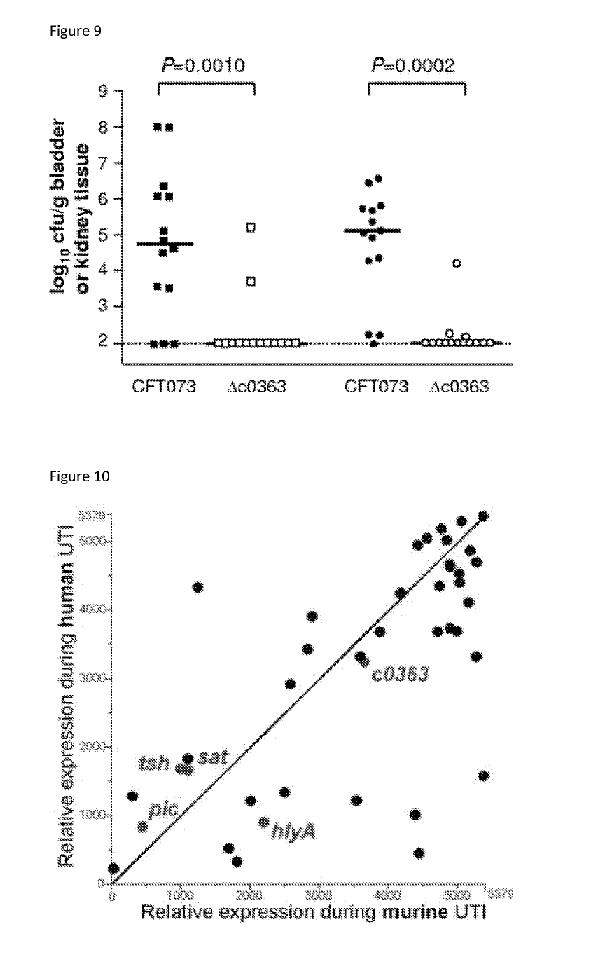

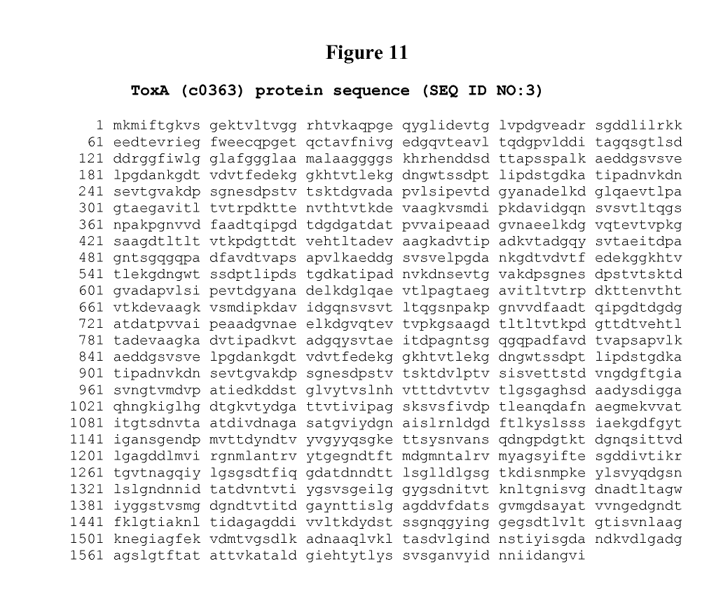

Discovery of an RTX exotoxin by deletion analysis of pathogenicity islands (PAI). UPEC Strain CFT073 contains 13 large genomic islands ranging in size from 32 kb to 123 kb. Eleven of these genomic islands were individually deleted from the genome, and nine isogenic mutants were tested for their ability to colonize the CBA/J mouse model of ascending UTI. Three genomic island mutants (ΔPAI-aspV, ΔPAImetV, and ΔPAI-asn7) were significantly outcompeted by wild-type CFT073 in the bladders and/or kidneys following transurethral cochallenge of CBA/J mice (P<0.039). A putative RTX family exoprotein encoded by tosA (c0363) within the PAI-aspV island contributed significantly to the observed phenotype. Two independent tosA deletion mutants were attenuated in the murine model (Lloyd et al., J. Bacteriol. 191:3469 2009); results for one of these mutants (FIG. 9) shows that the mutant was outcompeted by ˜700-fold in the bladder and ˜1000-fold in the kidney.

The putative RTX exotoxin was independently identified by in vivo-induced antigen technology (IVIAT). To identify UPEC genes expressed in vivo but not in vitro, sera from mice chronically infected with UPEC strain CFT073 were adsorbed 24 times with both in vitro-cultured CFT073 and in vitro cultured commensal strain BL21 to remove antibodies against proteins synthesized in vitro. Adsorbed sera were used against an expression library expressing CFT073 antigens by colony immunoblot. Immuno-reactive clones were identified, and expression library clones were sequenced. Ninety-three genes were identified as in vivo-expressed genes, including tosA (c0363). This putative RTX exotoxin is only expressed in vivo as assessed by qRT-PCR of mRNA isolated from bacteria recovered directly from the urine of infected mice; negligible expression was detected from in vitro-cultured UPEC CFT073.

Virulence gene assessment in an E. coli strain collection by multiplex PCR. A collection of 315 strains has been assembled that include, in increasing virulence, 91 fecal strains, 68 asymptomatic bacteriuria strains, 41 host-compromised strains, 37 cystitis strains, and 78 acute pyelonephritis strains. Primers, capable of PCR-amplifying segments of 15 different virulence genes, were developed. PCR products were of such size that 7-8 amplifications could be conducted simultaneously and identified in a single lane of agarose gels (multiplex PCR). The results for all 315 strains are summarized in Table 3. FIG. 10 shows that there is good concordance of toxin expression (especially for c0363 tosA) in both mice and women with UTI.

| | Fimbriae | | Toxins | | | | | | Iron Acquisition | | | | | | |
|---|---|---|---|---|---|---|---|---|---|---|---|---|---|---|---|
| Strain type (N)^{a} | fimA | papA | hlyA | cnf1 | c0363 | sat | pic | tsh | chuA | hma | iutA | iroN | fyuA | iha | ireA |
| Fecal (91) | 98.9 | 26.4 | 13.2 | 7.7 | 11.0 | 19.8 | 12.1 | 20.9 | 39.6 | 26.4 | 31.9 | 20.9 | 47.3 | 31.9 | 14.3 |
| Host comp. (41) | 97.6 | 51.2 | 19.5 | 4.9 | 19.5 | 41.5 | 12.2 | 48.8 | 75.6 | 34.1 | 58.5 | 26.8 | 78.0 | 46.3 | 24.4 |
| ABU (68) | 98.5 | 54.4 | 35.3 | 33.8 | 26.5 | 33.8 | 23.5 | 63.2 | 73.5 | 51.5 | 45.6 | 41.2 | 83.8 | 35.3 | 16.2 |
| Cystitis (37) | 97.3 | 67.6 | 48.6 | 32.4 | 16.2 | 32.4 | 18.9 | 54.1 | 83.8 | 81.1 | 43.2 | 48.6 | 89.2 | 37.8 | 10.8 |
| Pyelonephritis (78) | 98.7 | 63.6 | 48.1 | 13.0 | 28.6 | 51.9 | 29.9 | 67.5 | 89.6 | 61.0 | 66.2 | 41.6 | 93.5 | 55.8 | 27.3 |
| c0363⁺ strains (63) | 98.4 | 59.4 | 70.3 | 37.5 | 100 | 67.2 | 89.1 | 96.9 | 98.4 | 92.2 | 67.2 | 59.4 | 98.4 | 67.2 | 20.3 |
| c0363⁻ strains (252) | 98.4 | 47.2 | 21.6 | 12 | 0 | 26.8 | 2.0 | 36.8 | 61.6 | 36.4 | 43.2 | 28.0 | 69.6 | 34.4 | 18.4 |

^{a}N, number of isolates in each strain type; Host comp., Host compromised (catheter-associated UTI and vesicoureteral reflux); ABU, asymptomatic bacteriuria; c0363 is tosA encoding the putative RTX toxin

### EXAMPLE 3

### TosA (RTX) Based Vaccines

TosA is expressed in vitro and purified. The purified protein is used to intranasally immunize mice to test for protection against the development of UTI as described for the iron acquisition proteins (Alteri et al., (2009). PLoS Pathog 5, e1000586). CBA/J mice (N=20) are immunized (day 1; 100 μg) and boosted twice (day 14 and 21; 25 μg) with TosA conjugated to the adjuvant cholera toxin. As a control, mice (N=10) is immunized with cholera toxin alone (a total of 30 mice). At day 28, mice are transurethrally challenged with 1×10⁸ cfu CFT073. After 48 hr, urine, bladder, kidneys, and spleens are homogenized and quantitatively cultured. Data is analyzed for significance using the Mann-Whitney test (Alteri et al., 2009 PLoS Pathog 5, e1000586).

### EXAMPLE 4

### FyuA

This Example describes the FyuA antigen. A recent large-scale screening process to identify vaccine candidates against UPEC consistently identified proteins involved in iron uptake. UPEC strains carry an increased abundance and variety of iron acquisition systems in comparison to intestinal commensal E. coli. In the iron-limited environment of the human urinary tract, these additional iron acquisition systems facilitate successful colonization and have been shown to be highly expressed during human UTI.

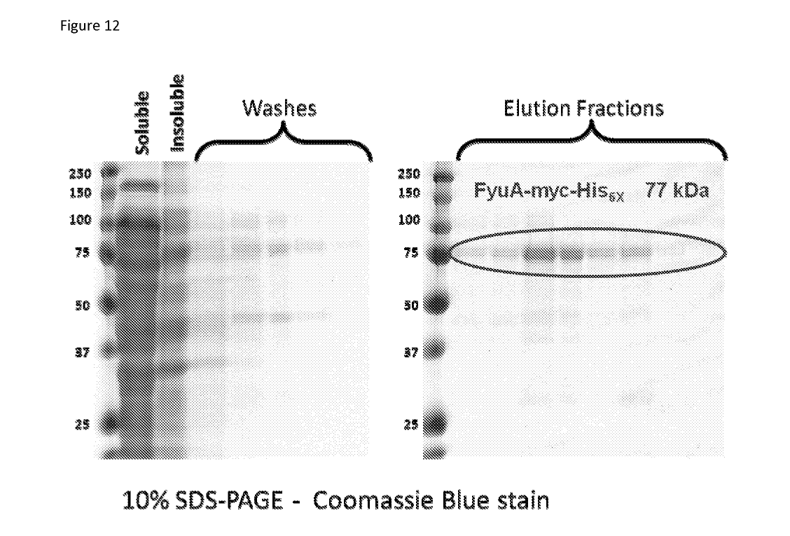

Using the murine model of ascending UTI, the potential of UPEC iron acquisition receptors to provide protective immunity from infection was assayed. Outer membrane iron acquisition receptors were cloned from UPEC strains CFT073 and 536 and purified as affinity-tagged recombinant proteins. Once purified (FIG. 12), proteins were chemically crosslinked to cholera toxin (CT) as adjuvant and administered intranasally. After primary and subsequent booster immunizations, mice were challenged with UPEC transurethrally and protective immunity was assessed 48 hours post-inoculation by quantifying colony forming units in the urine, bladder and kidneys.

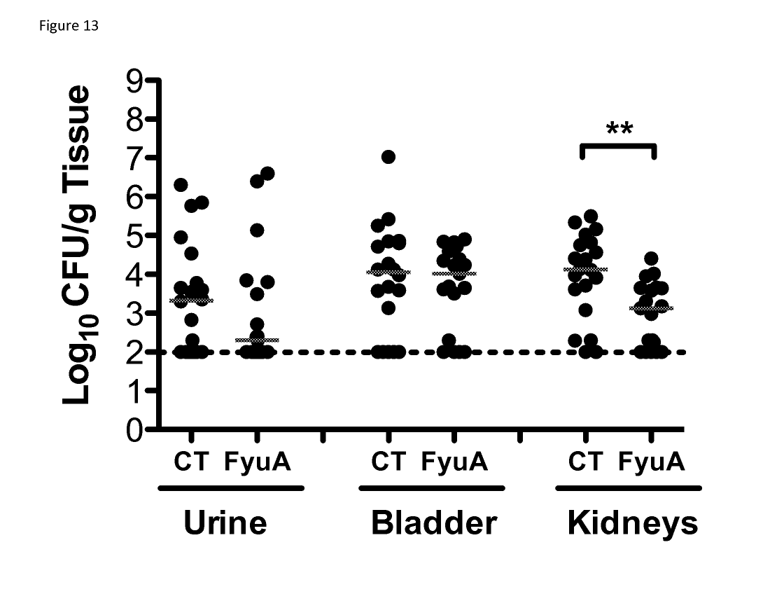

It was found that immunization with the yersiniabactin siderophore receptor FyuA provided significant protection from UPEC infection in the kidneys (p=0.0018) (FIG. 13) in contrast to the putative iron receptor c0294 and a heme receptor ChuA peptide fragment, neither of which was found to convey protection.

These data indicate that UPEC iron receptors are a class of vaccine targets for the prevention of UTI.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A vaccine composition comprising at least a portion of one or more immunogens selected from the group consisting of TosA and FyuA.

2. The vaccine composition of claim 1, wherein said vaccine composition further comprises one or more immunogens selected from the group consisting of Iha, IreA, IroN, IutA and c2482.

3. The composition of claim 1, wherein said immunogen is covalently bound to a carrier protein.

4. The composition of claim 1, wherein said carrier protein is selected from the group consisting of cholera toxin, pseudomonas exotoxin A, toxoids, virus like particles, tetanus toxin/toxoid, diphtheria toxin/toxoid and hepatitis B surface protein.

5. The composition of claim 3, wherein said carrier protein is a sterile pharmaceutically acceptable carrier protein.

6. The composition of claim 1, wherein said at least a portion is a peptide that corresponds to extracellular loop 7 of IroN or loop 6 of IutA.

7. A kit comprising the composition of claim 1.

8. The kit of claim 7, wherein said kit further comprises a device for administration of said vaccine.

9. The kit of claim 7, wherein said kit further comprises one or more additional components selected from the group consisting of sanitation components, temperature control components, adjuvants, a physiologically tolerable buffer and instructions for using said vaccine composition.

10. A method of inducing an immune response, comprising administering a composition comprising an effective amount of at least a portion of a TosA immunogen to a subject under conditions such that said subject generates an immune response to a bacteria in said subjects urinary tract.

11. The method of claim 10, wherein said immunogen is covalently bound to a carrier protein.

12. The method of claim 11, wherein said carrier protein is selected from the group consisting of cholera toxin, pseudomonas exotoxin A, toxoids, virus like particles, tetanus toxin/toxoid, diphtheria toxin/toxoid and hepatitis B surface protein.

13. The method of claim 11, wherein said carrier protein is a sterile pharmaceutically acceptable carrier protein.

14. The method of claim 10, wherein said composition further comprises at least a portion of one or more antigens selected from the group consisting of Iha, FyuA, IreA, IutA, IroN and c2482.

15. The method of claim 10, wherein said bacteria is E. coli.

16. The method of claim 10, wherein said composition further comprises an adjuvant.

17. The method of claim 10, wherein said immune response protects said subject from developing symptoms of a urinary tract infection.

18. The method of claim 10, wherein said subject exhibits decreased levels of bacteria in said subject's bladder or kidney.

19. A method of preventing urinary tract infections in a subject, comprising administering a composition comprising an effective amount of at least a portion of a Tos A immunogen to a subject under conditions such that said subject does not develop symptoms of a urinary tract infection.

20. The method of claim 19, wherein said subject exhibits decreased levels of bacteria in said subject's bladder or kidney.

21. A method of inducing an immune response, comprising administering a composition comprising an effective amount of at least a portion of a FyuA immunogen to a subject under conditions such that said subject generates an immune response to a bacteria in said subjects urinary tract.

22. The method of claim 21, wherein said composition further comprises at least a portion of one or more antigens selected from the group consisting of Iha, TosA, IreA, IutA, IroN and c2482.


SEQUENCE LISTING (103 sequences)

SEQ ID NO: 1 - DNA, 39 bp, Artificial Sequence
  Synthetic
nnnnnntacgtaatgaaaactcaaataactttcgctgcg

SEQ ID NO: 2 - DNA, 34 bp, Artificial Sequence
  Synthetic
nnnnnngagctcctatttttcgattgcaccatcc

SEQ ID NO: 3 - protein, 1610 aa, Escherichia coli
MKMIFTGKVSGEKTVLTVGGRHTVKAQPGEQYGLIDEVTGLVPDGVEADRSGDDLILRKKEEDTEVRIEGFWEECQPGETQCTAVFNIVGEDGQVTEAVLTQDGPVLDDITAGQSGTLSDDDRGGFIWLGGLAFGGGLAAMALAAGGGGSKHRHENDDSDTTAPSSPALKAEDDGSVSVELPGDANKGDTVDVTFEDEKGGKHTVTLEKGDNGWTSSDPTLIPDSTGDKATIPADNVKDNSEVTGVAKDPSGNESDPSTVTSKTDGVADAPVLSIPEVTDGYANADELKDGLQAEVTLPAGTAEGAVITLTVTRPDKTTENVTHTVTKDEVAAGKVSMDIPKDAVIDGQNSVSVTLTQGSNPAKPGNVVDFAADTQIPGDTDGDGATDATPVVAIPEAADGVNAEELKDGVQTEVTVPKGSAAGDTLTLTVTKPDGTTDTVEHTLTADEVAAGKADVTIPADKVTADGQYSVTAEITDPAGNTSGQGQPADFAVDTVAPSAPVLKAEDDGSVSVELPGDANKGDTVDVTFEDEKGGKHTVTLEKGDNGWTSSDPTLIPDSTGDKATIPADNVKDNSEVTGVAKDPSGNESDPSTVTSKTDGVADAPVLSIPEVTDGYANADELKDGLQAEVTLPAGTAEGAVITLTVTRPDKTTENVTHTVTKDEVAAGKVSMDIPKDAVIDGQNSVSVTLTQGSNPAKPGNVVDFAADTQIPGDTDGDGATDATPVVAIPEAADGVNAEELKDGVQTEVTVPKGSAAGDTLTLTVTKPDGTTDTVEHTLTADEVAAGKADVTIPADKVTADGQYSVTAEITDPAGNTSGQGQPADFAVDTVAPSAPVLKAEDDGSVSVELPGDANKGDTVDVTFEDEKGGKHTVTLEKGDNGWTSSDPTLIPDSTGDKATIPADNVKDNSEVTGVAKDPSGNESDPSTVTSKTDVLPTVSISVETTSTDVNGDGFTGIASVNGTVMDVPATIEDKDDSTGLVYTVSLNHVTTTDVTVTVTLGSGAGHSDAADYSDIGGAQHNGKIGLHGDTGKVTYDGATTVTIVIPAGSKSVSFIVDPTLEANQDAFNAEGMEKVVATITGTSDNVTAATDIVDNAGASATGVIYDGNAISLRNLDGDFTLKYSLSSSIAEKGDFGYTIGANSGENDPMVTTDYNDTVYVGYYQSGKETTSYSNVANSQDNGPDGTKTDGNQSITTVDLGAGDDLMVIRGNMLANTRVYTGEGNDTFTMDGMNTALRVMYAGSYIFTESGDDIVTIKRTGVTNAGQIYLGSGSDTFIQGDATDNNDTTLSGLLDLGSGTKDISNMPKEYLSVYQDGSNLSLGNDNNIDTATDVNTVTIYGSVSGEILGGYGSDNITVTKNLTGNISVGDNADTLTAGWIYGGSTVSMGDGNDTVTITDGAYNTTISLGAGDDVFDATSGVMGDSAYATVVNGEDGNDTFKLGTIAKNLTIDAGAGDDIVVLTKDYDSTSSGNQGYINGGEGSDTLVLTGTISVNLAAGKNEGIAGFEKVDMTVGSDLKADNAAQLVKLTASDVLGINDNSTIYISGDANDKVDLGADGAGSLGTFTATATTVKATALDGIEHTYTLYSSVSGANVYIDNNIIDANGVI

SEQ ID NO: 4 - DNA, 38 bp, Artificial Sequence
  Synthetic
acacagcgataaagtactcaaaagcctcgagactcacg

SEQ ID NO: 5 - DNA, 24 bp, Artificial Sequence
  Synthetic
cagtcggtagagcaggggattgaa

SEQ ID NO: 6 - DNA, 38 bp, Artificial Sequence
  Synthetic
tattgccatttccttaaccccacctgataacccttagc

SEQ ID NO: 7 - DNA, 27 bp, Artificial Sequence
  Synthetic
agcgactggagtttgggcgggggtagg

SEQ ID NO: 8 - DNA, 41 bp, Artificial Sequence
  Synthetic
caggctgatggtacatgctctgaaactggctgcaggatacg

SEQ ID NO: 9 - DNA, 27 bp, Artificial Sequence
  Synthetic
gcacagaaggaaagtacctggctatta

SEQ ID NO: 10 - DNA, 42 bp, Artificial Sequence
  Synthetic
tcgcttttactgaaattaggttgacgagatgtgcagattacg

SEQ ID NO: 11 - DNA, 27 bp, Artificial Sequence
  Synthetic
ggagatggttgctgaacgtgtggatta

SEQ ID NO: 12 - DNA, 41 bp, Artificial Sequence
  Synthetic
gtgcagttcccttctgaaaatacttaatcacaaacatctca

SEQ ID NO: 13 - DNA, 27 bp, Artificial Sequence
  Synthetic
tcagacaactcactcacctctcatctc

SEQ ID NO: 14 - DNA, 42 bp, Artificial Sequence
  Synthetic
ataacccatcagcccgcttctgtaatacctccattcgttcta

SEQ ID NO: 15 - DNA, 27 bp, Artificial Sequence
  Synthetic
tgaaattatactgaacggatacaagac

SEQ ID NO: 16 - DNA, 39 bp, Artificial Sequence
  Synthetic
gggcctctatcttcaatctgttcgactaacccctcctct

SEQ ID NO: 17 - DNA, 27 bp, Artificial Sequence
  Synthetic
ctttgtcgccacttgttttaccttaga

SEQ ID NO: 18 - DNA, 40 bp, Artificial Sequence
  Synthetic
agaatcattccatttcgaaatcattaatcttcacttcaag

SEQ ID NO: 19 - DNA, 27 bp, Artificial Sequence
  Synthetic
cgatggcgacaaattggcggcagagtc

SEQ ID NO: 20 - DNA, 41 bp, Artificial Sequence
  Synthetic
gtattgctgaagctgcacgtactgcccggataatgcgagag

SEQ ID NO: 21 - DNA, 27 bp, Artificial Sequence
  Synthetic
gtaaaggggcgggggaaatgggttttt

SEQ ID NO: 22 - DNA, 42 bp, Artificial Sequence
  Synthetic
cttttccctgaagagaccggatgtgatcgtccagatgaatag

SEQ ID NO: 23 - DNA, 27 bp, Artificial Sequence
  Synthetic
acaggaacaacaatttggtgaggtgtc

SEQ ID NO: 24 - DNA, 42 bp, Artificial Sequence
  Synthetic
cttcttttaacgttatcccaatggcatcacagcgtagtgtaa

SEQ ID NO: 25 - DNA, 27 bp, Artificial Sequence
  Synthetic
ttatagcgatcggtggttgcctggact

SEQ ID NO: 26 - DNA, 42 bp, Artificial Sequence
  Synthetic
ggcttcttccagtggtacgtaattttctccgtttcccgatac

SEQ ID NO: 27 - DNA, 27 bp, Artificial Sequence
  Synthetic
acgtggaacaaatagacacaagaaata

SEQ ID NO: 28 - DNA, 42 bp, Artificial Sequence
  Synthetic
ccttaccgacgcaaaaatccgcaccctcaagccttctgataa

SEQ ID NO: 29 - DNA, 27 bp, Artificial Sequence
  Synthetic
gccccgttctcacgattcctctgtagt

SEQ ID NO: 30 - DNA, 42 bp, Artificial Sequence
  Synthetic
cagcgtgattcttgcggtaccgaagcggcttaaccagtctgt

SEQ ID NO: 31 - DNA, 27 bp, Artificial Sequence
  Synthetic
gcattcgtgacgttcggcacatagttc

SEQ ID NO: 32 - DNA, 42 bp, Artificial Sequence
  Synthetic
acccccatatgtcccttaacgacgcaaaaatccgtagtctca

SEQ ID NO: 33 - DNA, 27 bp, Artificial Sequence
  Synthetic
gcatcgctaatattcgcctcgttctca

SEQ ID NO: 34 - DNA, 42 bp, Artificial Sequence
  Synthetic
ggaagcgctgatcctctcccctagtggaactgtgtctaaagn

SEQ ID NO: 35 - DNA, 27 bp, Artificial Sequence
  Synthetic
cgggaatgcctgtgcaaattagttctg

SEQ ID NO: 36 - DNA, 42 bp, Artificial Sequence
  Synthetic
ggttgacctaaggtagcagtttatcctgatgcgctgagattt

SEQ ID NO: 37 - DNA, 27 bp, Artificial Sequence
  Synthetic
gctgacatcatcaagaataaaaaggtt

SEQ ID NO: 38 - DNA, 40 bp, Artificial Sequence
  Synthetic
cacggaaacagaaggtgtggtggaattatgcgaagaggtt

SEQ ID NO: 39 - DNA, 23 bp, Artificial Sequence
  Synthetic
ttccggatgttgcagggcgtaat

SEQ ID NO: 40 - DNA, 42 bp, Artificial Sequence
  Synthetic
agtaaaccgttaatatccctccatcaaagccatccatcttat

SEQ ID NO: 41 - DNA, 24 bp, Artificial Sequence
  Synthetic
tttttcgtttttacgcttccttac

SEQ ID NO: 42 - DNA, 40 bp, Artificial Sequence
  Synthetic
ttgtaactgttaaatcaggcaaggcaatgtttgaagtagt

SEQ ID NO: 43 - DNA, 27 bp, Artificial Sequence
  Synthetic
tattagacgctggttttgtgactgatg

SEQ ID NO: 44 - DNA, 39 bp, Artificial Sequence
  Synthetic
aaaaactgatctgggggatgtagaaactcaaggaagtag

SEQ ID NO: 45 - DNA, 27 bp, Artificial Sequence
  Synthetic
tccttgatgctataggggtgctgagac

SEQ ID NO: 46 - DNA, 42 bp, Artificial Sequence
  Synthetic
atgacggtgagggagtagagtaatcaatcagttttagtgaat

SEQ ID NO: 47 - DNA, 27 bp, Artificial Sequence
  Synthetic
acccatttttccctctgcatactgttt

SEQ ID NO: 48 - DNA, 38 bp, Artificial Sequence
  Synthetic
ggagcggtagttcagtcggttagaatacctgcctgtca

SEQ ID NO: 49 - DNA, 25 bp, Artificial Sequence
  Synthetic
aacagcaacaaggtgaaacaacaat

SEQ ID NO: 50 - DNA, 39 bp, Artificial Sequence
  Synthetic
cgaaaaatcactaacgaaacattggatccccattgttgc

SEQ ID NO: 51 - DNA, 24 bp, Artificial Sequence
  Synthetic
ctgagagcgaggagcggaagtaag

SEQ ID NO: 52 - DNA, 38 bp, Artificial Sequence
  Synthetic
cagctttctgacaacccggccgctcctgctttcaacaa

SEQ ID NO: 53 - DNA, 28 bp, Artificial Sequence
  Synthetic
tacatgcttacttccgctcctcgctctc

SEQ ID NO: 54 - DNA, 38 bp, Artificial Sequence
  Synthetic
ggtttttaatgcacactggataacaaccggacgaatct

SEQ ID NO: 55 - DNA, 28 bp, Artificial Sequence
  Synthetic
tagtctgatatggcttttgtcgctgtcc

SEQ ID NO: 56 - DNA, 42 bp, Artificial Sequence
  Synthetic
tgataagccaaattgataagctggaatatgtgatgaaagtgc

SEQ ID NO: 57 - DNA, 28 bp, Artificial Sequence
  Synthetic
cagcgccagtgatatttgaagattcgtc

SEQ ID NO: 58 - DNA, 42 bp, Artificial Sequence
  Synthetic
cttgtcatatccggataaaatcaccctctggtaatactctta

SEQ ID NO: 59 - DNA, 28 bp, Artificial Sequence
  Synthetic
tgcacatcgcacaagtgattatgaacag

SEQ ID NO: 60 - DNA, 42 bp, Artificial Sequence
  Synthetic
gaattaagcgccagacgtatggtcaaaagtagtggagtagaa

SEQ ID NO: 61 - DNA, 28 bp, Artificial Sequence
  Synthetic
ttattgttaccttcttttgttgtgatga

SEQ ID NO: 62 - DNA, 38 bp, Artificial Sequence
  Synthetic
catggcgggatgcggatgagtttaggttgctgtgagtg

SEQ ID NO: 63 - DNA, 28 bp, Artificial Sequence
  Synthetic
ttgatgcggagttgtcgatggctgtatt

SEQ ID NO: 64 - DNA, 38 bp, Artificial Sequence
  Synthetic
agcctcccgttcgcttcacttaccgcctgctgagttac

SEQ ID NO: 65 - DNA, 24 bp, Artificial Sequence
  Synthetic
ttcactgccggactgcctctggtt

SEQ ID NO: 66 - DNA, 42 bp, Artificial Sequence
  Synthetic
ccgtagaaagccccactgcaccaacgccaaaaccaccattta

SEQ ID NO: 67 - DNA, 24 bp, Artificial Sequence
  Synthetic
agcgcaagcatcaataaaaatagt

SEQ ID NO: 68 - DNA, 37 bp, Artificial Sequence
  Synthetic
gggacttattgttgtctctgaacttgccagcagggta

SEQ ID NO: 69 - DNA, 24 bp, Artificial Sequence
  Synthetic
acatcatggagagtccgcagtgaa

SEQ ID NO: 70 - DNA, 37 bp, Artificial Sequence
  Synthetic
cgcatcacgcagtaccgtctcaccattattcagtagg

SEQ ID NO: 71 - DNA, 24 bp, Artificial Sequence
  Synthetic
gctgacttctcaaactccagacca

SEQ ID NO: 72 - DNA, 39 bp, Artificial Sequence
  Synthetic
gctcctcgctctcagcggaccttcagctcagtgatatcg

SEQ ID NO: 73 - DNA, 24 bp, Artificial Sequence
  Synthetic
ggggatccaatgtttcgttagtga

SEQ ID NO: 74 - DNA, 40 bp, Artificial Sequence
  Synthetic
cctgaggctgaagcaaccacgttaatccgactatttttcg

SEQ ID NO: 75 - DNA, 24 bp, Artificial Sequence
  Synthetic
tggtaggcggatagataatagaaa

SEQ ID NO: 76 - DNA, 32 bp, Artificial Sequence
  Synthetic
ctgtcggaggtcgtcatacagttaaagcacag

SEQ ID NO: 77 - DNA, 24 bp, Artificial Sequence
  Synthetic
taccgatggtgatggtgcaacaga

SEQ ID NO: 78 - DNA, 36 bp, Artificial Sequence
  Synthetic
gttaatatagccctgattaccggaggaggtggagtc

SEQ ID NO: 79 - DNA, 24 bp, Artificial Sequence
  Synthetic
atggatactttaccggcagccact

SEQ ID NO: 80 - DNA, 38 bp, Artificial Sequence
  Synthetic
aggaagaagatactgaggtccggatagagggattctgg

SEQ ID NO: 81 - DNA, 22 bp, Artificial Sequence
  Synthetic
tgtcggaggtcgtcatacagtt

SEQ ID NO: 82 - DNA, 38 bp, Artificial Sequence
  Synthetic
ccgttaatatagccctgattaccggaggaggtggagtc

SEQ ID NO: 83 - DNA, 22 bp, Artificial Sequence
  Synthetic
taacagttgttgccgttgccgt

SEQ ID NO: 84 - DNA, 38 bp, Artificial Sequence
  Synthetic
aaggatatggccgacagtttccagaatgaagttcccgc

SEQ ID NO: 85 - DNA, 24 bp, Artificial Sequence
  Synthetic
attctctcaccagataatgccgcc

SEQ ID NO: 86 - DNA, 38 bp, Artificial Sequence
  Synthetic
tattccttctgggcgcgaacgatagcctgtataaagcg

SEQ ID NO: 87 - DNA, 24 bp, Artificial Sequence
  Synthetic
atcaacagacgaagccagacagca

SEQ ID NO: 88 - DNA, 40 bp, Artificial Sequence
  Synthetic
cttttgggtgtggggttactgctccttgttgtgttgttgc

SEQ ID NO: 89 - DNA, 24 bp, Artificial Sequence
  Synthetic
cattgccaggcatcgtctttggtt

SEQ ID NO: 90 - DNA, 38 bp, Artificial Sequence
  Synthetic
caatgaatttatatttcgttgaatagataacatttacc

SEQ ID NO: 91 - DNA, 24 bp, Artificial Sequence
  Synthetic
acaggctgggtttgccgtactaaa

SEQ ID NO: 92 - DNA, 38 bp, Artificial Sequence
  Synthetic
agaaggcggtgcttcaatcacactaacaaggagagtaa

SEQ ID NO: 93 - DNA, 28 bp, Artificial Sequence
  Synthetic
cagtcaaccggcgtgtcgaaatcagtct

SEQ ID NO: 94 - DNA, 38 bp, Artificial Sequence
  Synthetic
gtggtgtgcgtggtcgaagaactgtacgttcataagag

SEQ ID NO: 95 - DNA, 28 bp, Artificial Sequence
  Synthetic
ctcgcggccttcaaaggtcagcacatcc

SEQ ID NO: 96 - DNA, 39 bp, Artificial Sequence
  Synthetic
gcagaacgaagcctcgacgatatcactatacgctcaacc

SEQ ID NO: 97 - DNA, 23 bp, Artificial Sequence
  Synthetic
tggggttactgctccttgttgtg

SEQ ID NO: 98 - DNA, 40 bp, Artificial Sequence
  Synthetic
ctgatttaaccgggtatcaatttgcgtcaacagcgttggc

SEQ ID NO: 99 - DNA, 20 bp, Artificial Sequence
  Synthetic
gcggtttgtcagcattctaa

SEQ ID NO: 100 - DNA, 37 bp, Artificial Sequence
  Synthetic
ttttgtgactgatgtcggatatttgaatgtcggcttg

SEQ ID NO: 101 - DNA, 22 bp, Artificial Sequence
  Synthetic
gacgctggcgagaaggggataa

SEQ ID NO: 102 - DNA, 37 bp, Artificial Sequence
  Synthetic
atctcccttcctgcgaagtaatcaattatcgactggg

SEQ ID NO: 103 - DNA, 24 bp, Artificial Sequence
  Synthetic
atttcggtagatagcttgggttcg